(19) United States
(12) Patent Application Publication
(10) Pub. No.: US 2016/0008777 A1
(43) Pub. Date: Jan. 14, 2016

(54) PHARMACEUTICAL COMPOUNDING KIT

(71) Applicant: Novotec Consulting Inc., East Windsor, NJ (US)

(72) Inventors: Kunal PATEL, Old Bridge, NJ (US); David F. ERKOBONI, Pennington, NJ (US); Snehal PATEL, Old Bridge, NJ (US); Piushbhai J. PATEL, Monroe Township, NJ (US); Lyndsey Marie SZEM, Wall, NJ (US); Jeimy RODRIGUEZ, South Brunswick, NJ (US)

(21) Appl. No.: 14/794,008

(22) Filed: Jul. 8, 2015

Related U.S. Application Data
(60) Provisional application No. 62/022,374, filed on Jul. 9, 2014.

(51) Int. Cl. B01F 11/00; A61K 31/714; A61K 31/351; A61K 31/569; A61K 31/167; A61K 31/436; A61K 38/48; A61K 31/7036; A61K 31/4166; A61K 31/4412; A61K 31/485; A61K 31/245; A61J 3/04; A61J 3/08; A61J 3/07; A61K 31/495

(52) U.S. Cl. CPC B01F 11/0005; A61K 31/495; A61K 31/714; A61K 31/351; A61K 31/569; A61K 31/167; A61K 31/436; A61K 38/4886; A61K 31/7036; A61K 31/4166; A61K 31/4412; A61K 31/485; A61K 31/245; A61J 3/04; A61J 3/08; A61J 3/07; C12Y 304/24007; B01F 2215/0032

(57) ABSTRACT

A kit which is useful in the preparation of a compounded pharmaceutical product is provided. A method for using such a kit to make a compounded pharmaceutical product, and a compounded pharmaceutical product are also provided. Further provided is an apparatus for use in making a compounded pharmaceutical product.

### CROSS REFERENCE TO RELATED APPLICATIONS

The present application claims the priority of U.S. provisional Application No. 62/022,374, filed Jul. 9, 2014, the content of which is incorporated herein by reference in its entirety.

### FIELD OF THE INVENTION

The present invention relates to a kit which is useful in the preparation of a compounded pharmaceutical product. The present invention also relates to a compounded pharmaceutical product and methods for making the same. The present invention further relates to an apparatus for use in the preparation of a compounded pharmaceutical product.

### BACKGROUND OF THE INVENTION

Pharmaceutical compounding is the preparation of a pharmaceutical product to suit the personalized needs of a patient. Compounding may achieve this through various means. For example, compounding may place a pharmaceutically active agent into a desired dosage form so that preferred routes of administration may be used. Compounding may also be done to achieve a customized dosage strength for the pharmaceutically active agent. Further, compounding may allow for avoidance of ingredients that may cause an adverse effect in a patient. In addition, compounding may add a desired flavor to a drug form. Compounding may also combine two or more pharmaceutically active agents into one dosage form so that they may be administered together. This allows for better convenience to the patient and better patient compliance. In particular, compounding may be used to combine two or more pharmaceutically active agents that are normally incompatible with each other into a dosage form in which they can be administered together. Each of the above means and other means not discussed above may be conducted independently or in combination with one another during the process of compounding to prepare a pharmaceutical product to suit the personalized needs of a patient.

Compounding is typically done by a pharmacist and is generally considered a tedious, laborious and sometimes inconsistent process. As such, there is a need for a pharmaceutical compounding kit to assist pharmacists to more efficiently and consistently prepare compounded pharmaceutical products. However, pharmaceutical compounding kits known in the art typically contain pre-measured amounts of pharmaceutically active and pharmaceutically-inactive agents and are designed for combining fixed amounts of such agents to produce compounded pharmaceutical products having a pre-determined set dosage strength. Due to the prescription-specific nature of compounding, with different patients often requiring different dosage strengths, such kits are impractical as they do not allow for the production of a final product that is tailored to meet the needs of a specific patient's prescription. As a result, such kits are not typically used in the practice of compounding. There is therefore a need for a more versatile pharmaceutical compounding kit that contains pre-measured amounts of pharmaceutically active and pharmaceutically-inactive agents and yet is capable of producing compounded pharmaceutical products having various differing dosage strengths. This need is currently even more acute as health care is becoming increasingly personalized. In particular, there is a need for a pharmaceutical compound kit that can produce multiple unit of use amounts of the compounded pharmaceutical product with each unit of use potentially being of a differing dosage strength.

Embodiments of the kit of the present invention meet the above need in that their design allows for a metered amount of certain pharmaceutically active agent(s) as well as inactive agent(s) to be included in a compounded pharmaceutical product. As such, compounded pharmaceutical products of multiple active agents, each at various strengths (e.g., full strength, half strength, quarter strength and others) may be made using the same pharmaceutical compounding kit. This also allows for better mass production of a kit that can suit the needs of various patients, fulfill various prescriptions, and allow for customized medicines. This further allows for the production of multiple unit of use amounts of the compounded pharmaceutical product.

In addition to the above, embodiments of the kit of the present invention are capable of producing products of high uniformity, reproducibility and/or stability.

### SUMMARY OF THE INVENTION

The present invention relates in part to a kit for use in the preparation of a compounded pharmaceutical product comprising: a container which contains a pharmaceutically active agent, said kit being capable of producing compounded pharmaceutical products wherein the pharmaceutically active agent(s) can be present in said product at various dosage strengths.

In a particular embodiment, the kit also comprises a container which contains a pharmaceutically inactive agent.

In yet another particular embodiment, the kit comprises at least two containers which each contain a pharmaceutically active agent. In certain embodiments, each container individually contains an active agent that may be the same or different from those of another container.

In a further embodiment, at least one of the containers is in the form of a media dispenser which is capable of drawing and dispensing a metered amount of the contents therein.

In yet a further embodiment, the kit comprises a multi-chambered pouch wherein at least one chamber is a container containing a pharmaceutically active agent and another chamber is a container containing at least one pharmaceutically inactive agent, wherein the barrier between said containers is frangible or contains a frangible element and said kit further comprises a media dispenser which is capable of drawing and dispensing metered amounts of media.

In yet another particular embodiment, the invention relates to an apparatus for use in the production of a compounded pharmaceutical product, said apparatus comprising a housing which contains at least two chambers, an additional mixing chamber comprising a mixing means, and a dispensing means.

In a further embodiment, the invention relates to a kit comprising the aforementioned apparatus.

The present invention also relates to a method for making a compounded pharmaceutical product using a kit according to the present invention.

The present invention additionally relates to a compounded pharmaceutical product made using the aforementioned method.

### BRIEF DESCRIPTION OF THE DRAWINGS

FIG. 1 depicts a two-chambered pouch according to the present invention.

FIG. 2 depicts a pouch according to the present invention which comprises a chamber which serves as an empty discharge chamber.

### DETAILED DESCRIPTION OF THE INVENTION

The present invention is directed in part to a kit which is useful in the preparation of a compounded pharmaceutical product. In a particular aspect, the present invention relates to a kit which is capable of producing compounded pharmaceutical products wherein the pharmaceutically active agent can be present in said product at various dosage strengths. In another particular aspect, the present invention relates to a kit which is able to produce compounded pharmaceutical products reproducibly and/or uniformly. In yet another aspect, the present invention relates to a kit which is able to produce multiple unit of use amounts of a compounded pharmaceutical product.

Hereinafter in the specification and the claims, the following terms will have the following meanings.

The terms “active agent”, “active pharmaceutical ingredient”, “API”, and “pharmaceutically active agent” refer to a compound that is capable of producing a therapeutic effect in a subject to which it has been administered.

The terms “inactive agent”, “pharmaceutically inactive agent”, and “excipient” refer to a compound that is not capable of producing a therapeutic effect in a subject to which it has been administered. Instead, inactive agents are often used to form a dosage form in which the active agent can be administered (e.g., cream, ointment, gel, suppository, suspension, solution, tablet, capsule, spray, etc.) or to impart desired properties to the dosage form (e.g., anti-foaming agents, humectants, bulking agents, gelling agents, flavoring agents, etc.).

The term “base composition” refers to a composition comprising inactive agent(s) used to form a base for a compounded pharmaceutical product. A “base” is understood to be a vehicle used to convey or deliver the active agent(s).

The term “non-base inactive agent” refers to an inactive agent that does not form part of the base composition as initially supplied. Such agents (e.g., solubilizers, suspending agents) may be intended to be mixed with an active agent prior to the mixture of that active agent and the base composition.

The term “container” refers to any structure capable of containing an active and/or inactive agent such that it may not freely physically contact elements outside of said structure. Containers may also be of any size or shape and can be formed from any material. Containers may also be rigid or flexible. Examples of containers which may be used in the present invention include, but are not limited to: boxes, cylinders, vials, bottles (e.g., pump bottles), beakers, flasks, envelopes, pouches, sachets, sleeves, jars, blister packages, packets, tubes, media dispensers (such as syringes, pipettes, droppers, piston injectors, pipettors, spray bottles, spray nozzles, etc.), chambers within a pouch, cups, capsules, ampules, an evacuated container, and voids within a structure. Containers may be completely separate from each other. Alternatively, two or more containers may be contained in the same housing and/or may be separated from each other by a common barrier (e.g., a wall, sheet, film, membrane, or a seal). In certain embodiments, the common barrier may be frangible (e.g., removable or rupturable) or may contain an element (e.g., a seal) that is frangible, thus allowing the contents contained in the adjoining containers, following the fragmentation of the barrier or a frangible element therein, to mix with each other. In certain embodiments, the common barrier may be slideable, thus allowing the contents contained in the adjoining containers, following the sliding of the barrier to allow for a connection between the containers, to mix with each other.

In a particular embodiment, a common frangible barrier is formed by portions of the inner walls of the pouch which attach to one another (e.g., by adhering to each other), thus forming two or more compartments within the pouch with each compartment being capable of containing an active and/or inactive agent such that it may not freely physically contact elements outside thereof.

In yet another particular embodiment, two or more containers may be formed by crimping a first container, for example by using a mechanical clip. In such an embodiment, when said first container is crimped, two or more compartments are formed therein, with each compartment being capable of containing an active and/or inactive agent such that it may not freely physically contact elements outside thereof. As such, each compartment forms a container and the crimped areas serves as a common frangible barrier. In such an embodiment, the undoing of said crimping, for example by releasing or removing a mechanical clip, allows for the contents of the containers to mix.

Containers may contain amounts of a solid, semi-solid or liquid substance, for example, an active agent or an inactive agent. The term “active agent container” refers to a container which, as initially supplied in the kit, contains at least one active agent. In certain embodiments, the active agent container also contains at least one inactive agent. In certain other embodiments, the active agent container contains no inactive agents. In certain embodiments, the active agent container may contain two or more active agents. In certain other embodiments, the active agent container contains one active agent. The term “inactive agent container” refers to a container which, as initially supplied in the kit, contains at least one inactive agent but does not contain any active agent. In certain embodiments, an inactive agent container may contain two or more inactive agents. In certain other embodiments, the inactive agent container contains one inactive agent. The term “recipient container” refers to a container which, as initially supplied in the kit, does not contain any active or inactive agents; however, such a container may receive active and/or inactive agents during the course of the use of the kit for pharmaceutical compounding. It is understood that, in the practice of compounding using the kit of the present invention, inactive agents may later be added to active agent containers, active agents may later be added to inactive agent containers, additional active agents may later be added to active agent containers, additional inactive agents may later be added to inactive agent containers, and active and inactive agents may later be added to recipient containers.

The term “base container” refers to an inactive agent container that, as initially supplied in the kit, contains a base composition. A base container may additionally contain active agent(s) and/or inactive agent(s). A base container may optionally be marked with a fill line or fill lines such that, in embodiments in which a replacement base (discussed below) may need to be added to bring the final product to a desired target volume, the fill line(s) serve as a guide as to how much replacement base or other non-base inactive(s) should be added to achieve the target volume.

The term “non-base inactive container” refers to an inactive agent container that, as initially supplied in the kit, contains a non-base inactive agent but does not contain a base composition. In certain embodiments, non-base inactive containers may be containers which are graduated to allow for the drawing and/or dispensing of the contents therein in metered amounts (e.g., media dispensers, discussed below).

The term “associated containers” refers to two or more containers which contain active and/or inactive agents which are intended to be mixed with each other. For example, an active agent container containing an active agent may be associated with an inactive agent container containing a solubilizer which is capable of solubilizing the active agent. Likewise, for example, a container containing an active agent may be associated with an inactive agent container containing a suspending agent capable of suspending the active agent. An active agent container may be associated with a single inactive agent container or two or more inactive agent containers. For example, an active agent container may be associated with one inactive agent container which contains a solubilizer for the active agent and another inactive agent container which contains an anti-foaming agent for use with the active agent. Also, an inactive agent container may be associated with two or more active agent containers. For example, an inactive agent container may contain a solubilizer which is useful for solubilizing the active agents contained in two separate active agent containers. In a particular example, the active agent in an active agent container may be lipophilic. In such a case, said active agent container may be associated with an inactive agent container containing a lipophilic inactive agent, for example a lipophilic solubilizer. In another particular example, the active agent in an active agent container may be hydrophilic. In such a case, said active agent container may be associated with an inactive agent container containing a hydrophilic inactive agent. In certain embodiments, two or more active agent containers may be associated with each other, for example, when the active agents contained therein are intended to be mixed before they are added to a base composition. Also, in certain embodiments, two or more inactive agent containers may be associated with each other, for example, when an inactive agent container contains an inactive agent that is to be added to an associated base container (which is also an inactive agent container) or when the inactive agents contained in the associated inactive agent containers, when mixed, together form a base composition. In certain embodiments, each container may be coded to allow for a user to determine which containers are associated with each other.

In some cases, after the addition of active agent(s) and, if applicable, non-base inactive agent(s) to the base composition, it may be desirable to add additional inactive agents to the base composition to bring the final composition up to a desired volume. For example, in instances where less than the full amount of active agent(s) and/or non-base inactive agent(s) are added to a base composition (e.g., when less than full strength products are made), additional inactive agents may be added to bring the composition up to the same volume or weight that it would have had, had the full amount of the active agent(s) and/or non-base inactive agent(s) been added. In such instances, the inactive agents added may be additional base composition amounts, components of the base composition, non-base inactive agents (including those not previously added to the base composition), or mixtures thereof. For purposes of this specification and the claims, these inactive agents, when present in a kit for use in such a manner, will be called the “replacement base” and the inactive agent container which, as initially supplied in the kit, contains such inactive agents will be called the “replacement base container”. As discussed above, the replacement base container may contain additional base composition amounts. To alleviate confusion, in embodiments in which there is a replacement base container, the base container that does not contain the replacement base will be also referred to as the “primary base container”. It is contemplated that any container may be used as a replacement base container. For example, the replacement base container may be a media dispenser (described below) which may be used to transfer the contents of the replacement base container into the primary base container. In another embodiment, the replacement base container may be a pouch which contains the replacement base.

The term “connector means” refers to a means for connecting one container with another container such that the interior spaces of the containers are connected so that they are capable of forming one continuous space such that a liquid, solid, semi-solid, or gas can travel from one container to another.

The term “connector element” refers to any element which may be used to connect one container with another container such that the interior spaces of the containers are connected so that they are capable of forming one continuous space such that a liquid, solid, semi-solid, or gas can travel from one container to another. Examples of such connector elements include, but are not limited to: adapters (e.g., vial adapters, including needleless vial adapters), Luer locks, slip tips (e.g., Luer slips), valves (such as, for example, a needle valve, ball valve, butterfly valve, diaphragm valve, gate valve, globe valve, or plug valve), stopcocks, hollow needles (e.g., hypodermic needles), and combinations and portions thereof.

The term “mixing means” refers to a means for mixing the contents of a container.

The term “mixing element” refers to any element which may be used to mix compounds. Examples of such mixing elements include, but are not limited to: spatulas, tongue depressors, paddles, mechanical mixers, mechanical stirrers, stir bars, media dispensers, solid particles that aid in mixing the compounds (e.g., mixing balls), overhead mixers, and static mixers.

The term “dispensing means” refers to a means for dispensing the contents of a container.

The term “dispensing element” refers to any element which may be used to dispense the contents of a container. Examples of such dispensing elements include, but are not limited to: pumps, including metered pumps, volume calibrated caps, volume calibrated dispense discharge, length calibrated dispense discharge, pistons, hypodermic needles, bifurcated needles, nozzles, tubings, Luer fittings (such as Luer locks or Luer slips), catheters, auto injectors, jet injectors, syrettes, sprayers, droppers, burettes, pen injectors, a dispensing outlet, and a port which allows for contents of the container to be released.

The term “media dispenser” refers to a device which is capable of drawing and dispensing an amount of material, including but not limited to a liquid, a semi-solid, or powder. Examples of media dispensers include, but are not limited to: syringes, syrettes, pipettes, droppers (e.g., eye droppers), piston injectors, pipettors, burettes, tubes, pumps (including those with a length of discharge tubing), powder shakers, squeeze bottles, spray bottles, spray nozzles, and the like. As discussed above, the media dispenser may serve as a container. In such embodiments, it may be an active agent container, an inactive agent container, or a recipient container. In certain embodiments, the media dispenser is capable of drawing a metered amount of liquid, semi-solid, or powder and dispensing a metered amount of the contents therein.

The kit according to the present invention comprises at least one active agent container. In certain embodiments, the kit also comprises at least one inactive agent container. In certain embodiments, the containers may each individually contain pre-measured amounts of the agents therein.

The present invention is also directed to a method for making a compounded pharmaceutical product using a kit according to the present invention which comprises an active agent container and an inactive agent container. The method comprises the step of mixing an active agent from said active agent container with an inactive agent from said inactive agent container.

In certain embodiments of the present invention, the kit contains two or more active agent containers and/or two or more inactive agent containers.

In kits containing two or more active agent containers, each active agent container individually contains an active agent that may be the same or different from those of another active agent container. In certain embodiments, each container may contain a formulation comprising an active agent and optionally further comprising inactive agent(s). In particular embodiments, at least one of such containers contains a commercial formulation comprising an active agent. In such embodiments, the container may be the container in which the commercial formulation is sold in commercially.

In kits containing two or more inactive agent containers, each inactive agent container individually contains an inactive agent that may be the same or different from those of another inactive agent container.

The kit may further comprise a mixing means. Essentially any means capable of mixing the contents in a container may be used. In an embodiment of the present invention, the mixing means is a mixing element. Essentially any element capable of mixing the contents in a container may be used. Examples of such mixing elements include, but are not limited to: spatulas, tongue depressors, paddles, mechanical mixers, mechanical stirrers, stir bars, media dispensers, solid particles that aid in mixing the compounds (e.g., mixing balls), overhead mixers, and static mixers. The mixing means may be contained in or incorporated into such a container or may be separate from any container. In certain embodiments wherein the mixing means is contained in or incorporated into said container, the mixing means may be removable from the container and thus, for example, may be removed from the container after the contents thereof are mixed. In a particular embodiment, the mixing means are solid particles that aid in mixing the compounds (e.g., mixing balls) that may be added to a container. Shaking such a container causes the particles to mix the contents therein. Preferably, such particles do not react with the drug product.

The kit may also comprise a dispensing means. Essentially any means capable of dispensing the contents of a container may be used. In an embodiment of the present invention, the dispensing means is a dispensing element. Essentially any element capable of dispensing the contents of a container may be used. Examples of such a dispensing element include, but are not limited to: pumps, including metered pumps, volume calibrated caps, volume calibrated dispense discharge, length calibrated dispense discharge, pistons, hypodermic needles, bifurcated needles, injection ports, nozzles, tubings, Luer fittings (such as Luer locks or Luer slips), catheters, auto injectors, jet injectors, syrettes, sprayers, droppers, burettes, pen injectors, a dispensing outlet, and a port which allows for the contents of the container to be released. The dispensing means may be contained in or incorporated into such a container or may be separate from any container. In certain embodiments wherein the dispensing means is separate from any container, it may be attached to or otherwise incorporated into a container, for example an inactive agent container or a recipient container, prior to its use in dispensing the contents of the container. For example, the dispensing means may be an attachable pump such as a pump top. In an embodiment, the dispensing means is capable of dispensing a predetermined quantity of the contents thereof, for example a metered pump.

The kit may also comprise a connector means. Essentially any means capable of connecting one container with another container such that the interior spaces of the containers are connected so that they are capable of forming one continuous space such that a liquid, solid, semi-solid, or gas can travel from one container to another may be used. In an embodiment of the present invention, the connector means is a connector element. Essentially any element capable of connecting one container with another container such that the interior spaces of the containers are connected so that they are capable of forming one continuous space such that a liquid, solid, semi-solid, or gas can travel from one container to another may be used. In an embodiment, the connector means is capable of forming a connection that is airtight, vented, and/or aseptic. Examples of such connector means include, but are not limited to: adapters (e.g., vial adapters, including needleless vial adapters), injection ports, Luer locks, slip tips (e.g., Luer slips), barbs, valves (such as, for example, a needle valve, ball valve, butterfly valve, diaphragm valve, gate valve, globe valve, or plug valve), stopcocks, spikes, hollow needles (e.g., hypodermic needles), and combinations and portions thereof. The connector means may be contained in or incorporated into a container or may be separate from any container. In certain embodiments wherein the connector means is separate from any container, it may be attached to or otherwise incorporated therein. In certain embodiments, the containers to be connected may each contain, or be made to contain, a connector means which constitutes a corresponding fitting of the connector means contained, or to be contained, on the other. For example, one container may contain a female Luer lock fitting and the other container may contain a male Luer lock fitting. In embodiments in which the connector means is a hollow needle, the needle may be attached to or part of a container, for example, a media dispenser, and serve as a connector means when it is injected directly into another container.

In certain embodiments of the present invention, the container itself is of a form that allows for it to dispense its own contents and/or draw in contents, for example the contents of another container. Examples of such containers include, but are not limited to: bottles containing a pump, media dispensers, evacuated containers, and containers with a connector element, as described above. In such embodiments, it is contemplated that such a container may also be used to directly dispense the contents therein for use by a patient. In certain embodiments, the container is capable of dispensing a metered amount of the contents therein. An example of a container which is capable of dispensing a metered amount of the contents therein is a bottle with a metered pump. In certain embodiments, the container is capable of drawing and dispensing a metered amount of media. For example, the container may be in the form of a media dispenser which may draw a metered amount of a mixture from one container and also dispense a metered amount of the contents therein.

In a specific embodiment, the container is made at least partially of a material or materials which allow for it to occlude light. Examples of such materials include, but are not limited to, metallic foils, polymers or glass impregnated with materials that absorb light, including specifically the light of certain wavelengths (e.g., materials that absorb UV light or specific wavelengths of visible and infrared light), pigments (e.g., titanium dioxide and zinc oxide), amber glass, plastics, and films made from the same which may be layered on top of materials forming the container.

In an embodiment of the present invention, the kit comprises a housing which is capable of containing some or all of the elements of the kit (e.g., containers, mixing elements, dispensing elements, etc.). The housing may contain all of the elements of the kit or may contain some of the elements of the kit and not others. Essentially any housing capable of containing at least one element of the kit may be used. The housing may be of any form capable of containing at least one element of the kit and may be made from any material which is capable of containing at least one element of the kit. For example, the housing may be a box, a jar, or a plastic enclosure. In addition, a housing containing some elements of a kit may itself be contained in a housing which contains that housing and optionally additional elements of the kit. The housing may also contain multiple housings which each contain elements of the kit.

In one embodiment, the housing is in the form of a chambered pouch with the containers each forming a chamber within the pouch. Such an embodiment is described more extensively below. In another embodiment, the housing is made of an air-tight impenetrable material wherein voids within the housing form chambers which serve as the containers of the present invention. Such embodiments will be described more extensively below.

In an embodiment, the housing contains all the elements of the kit. Such a housing may contain at least one housing which itself contains containers, such as a pouch or a material containing voids which serve as containers, as described above.

In an embodiment of the present invention, the total volume occupied by the inactive agent or agents in the inactive agent container is equal to or less than the total volume of the container minus the total volume of the active agent(s) in the associated active agent container(s).

In an embodiment of the present invention, the total volume of all the inactive agents in all the inactive agent containers, including the base container, in a kit is equal to or less than the total volume of the base container minus the total volume of all the active agents in the kit.

The kit may comprise any number of non-base inactive containers, active agent containers, and base containers, and each individual container may be associated with a plurality of other containers. For example, it is contemplated that it may be that a plurality of non-base inactive containers may be associated with one active agent container, in other words each of the inactive agents contained in such containers may be intended to be mixed with the active agent in the associated active agent container. It is also contemplated that a plurality of active agent containers may be associated with one base container, in other words each of the active agents contained in such containers may be intended to be mixed with the base composition contained in the base container. In addition, it is contemplated that a plurality of active agent containers may be associated with one non-base inactive container. In an embodiment of the present invention, the kit comprises an active agent container that is associated with at least two inactive agent containers, with at least one of such containers being a base container and another one of such containers being a non-base inactive container.

In an embodiment of the present invention, the total volume occupied by the base composition in the base container is equal to or less than the total volume of the container minus the total volume of the active agent(s) contained in the associated active agent container(s) and the inactive agent(s) contained in the non-base inactive containers associated with such active agent container(s).

In an embodiment of the present invention, the kit comprises at least one active agent container, at least one inactive agent container, and a connector means, such as a connector element (e.g., a vial adapter, hollow needle), for connecting two or more containers. The connector means may be incorporated into a container or may be separate but intended to be incorporated into a container. The kit may also comprise a recipient container in the form of a media dispenser. In particular embodiments, both the active agent container and the inactive agent container are vials and the recipient container is a syringe. In another embodiment, at least one container is an evacuated container. In certain embodiments, the kit may comprise two or more inactive agent containers, for example, one container may be a base container and another container may be a non-base inactive container. In an example of such an embodiment, only the non-base inactive container(s) are to be connected to the active agent container(s) and a recipient container in the form of a media dispenser is used to transfer the contents of the containers into the base container. The recipient container may also be connected to the active agent container or the non-base inactive container by way of a connector means. Examples of such connector means include vial adapters and hollow needles. In embodiments in which the connector means is a hollow needle, the needle may be attached to or part of a media dispenser and serves as a connector means when it is injected directly into a container (e.g., through a rubber stopper covering a vial). A single connector means may connect all three containers (e.g., a three-way vial adapter or a two-way vial adapter with a Luer lock for attaching to a media dispenser). In another embodiment, the kit may comprise a further recipient container into which the contents of the other containers (including, in some embodiments, the base container) may be transferred, for example by way of the media dispenser. The mixing means may be contained in a container, incorporated into a container, or separate from any container. Non-limiting examples of mixing means for use in the present invention include: solid particles that aid in mixing the compounds (e.g., mixing balls) which are typically contained in the base container or may be in the kit separate from any container but then later added to the base container; an overhead mixer which may be separate from the container but used to mix the contents within the container; and a mechanical stirrer which is typically incorporated into the base container or may be in the kit separate from any container but then later added to the base container. The kit may also comprise a dispensing means which is associated with a base container, a recipient container, or is separate from any container. In the latter case, the dispensing means may be incorporated into a container or attached thereto prior to its use. An example of such a dispensing means is a metered pump. The kit may additionally comprise a replacement base container, in which case the previously mentioned base container would be the primary base container. The kit may further contain a mixing means, such as a mixing element. Each of the containers may individually be closed and airtight prior to their use. For example, a container which is a vial may be covered by a rubber stopper and a media dispenser may be covered with a cap. In an embodiment, the kit comprises a plurality of containers, for example two or more non-base inactive containers, two or more active agent containers, one primary base container, and a replacement base container. In an example of such an embodiment, each non-base inactive container may be associated with a specific active agent container and all active agent containers are associated with the single primary base container. In another such example, more than one non-base inactive container may be associated with a specific active agent container and all active agent containers are associated with the single primary base container. In yet another such example, a non-base inactive container may be associated with two or more active agent containers and all active agent containers are associated with the single primary base container.

In the practice of a method for making a compounded pharmaceutical product using the above kit, the inactive agent container is connected to an associated active agent container, for example, by a connector means. The contents of one container are then allowed to mix with the contents of another container. The container or containers containing the mixture may then be, for example, shaken or kneaded to further mix the contents therein. For example, a container containing a solubilizer and an active agent may be shaken or kneaded until the active agent is fully solubilized and a container containing a fluid containing a suspending agent and the active agent may be shaken or kneaded until a suspension of the active agent is achieved. Then, a recipient container in the form of a media dispenser is connected to the container(s). In certain embodiments, the media dispenser is connected to the same connector means that connects the active agent container and the inactive agent container (e.g., connected by a three-way connector means). The media dispenser may then optionally be used to mix the contents therein by drawing and dispensing the contents. The media dispenser is then used to draw either the full amount of the contents of the resulting mixture or a smaller metered amount thereof into the media dispenser. The media dispenser can then be used to dispense either the full amount of the contents thereof or a smaller metered amount. In embodiments wherein the kit further comprises a base container or a further recipient container, the media dispenser can be used to dispense either the full amount of the contents thereof or a smaller metered amount into said container. The aforementioned steps may be repeated or conducted in parallel if there are additional active agent containers and associated non-base inactive containers. In embodiments in which the media dispenser dispenses the contents thereof into a recipient container, the contents of a base container may also be dispensed into the recipient container. The kit may additionally comprise a replacement base container, in which case the previously mentioned base container would be the primary base container. In certain embodiments wherein a less than full metered amount of the active agent and non-base agent mixture is dispensed into a primary base container or a recipient container to which the base composition is added, the contents of a replacement base container may be added to said primary base container or recipient container to bring the amount of the compounded pharmaceutical up to a required amount. The kit may comprise a mixing means, in which case the mixing means may be used to mix the contents of the base container which now also contain the active agent(s) and the non-base inactive agent(s). For example, if the mixing means are solid particles that aid in mixing the compounds (e.g., mixing balls) contained in the base container, the container may be shaken to cause the particles to mix the contents therein. A dispensing means, either incorporated into the base container or added thereto is then used to dispense the resulting compounded pharmaceutical product.

In another embodiment of the present invention, the kit comprises at least one active agent container and at least one inactive agent container, and one of these containers is in the form of a media dispenser, for example a syringe. The other container may be, for example, in the form of a vial. The kit may further contain a connector means, such as a connector element, for connecting two or more containers. The connector means may be incorporated into a container or may be separate but intended to be incorporated into a container. Examples of such connector means include vial adapters, Luer lock fittings, and hollow needles. In embodiments in which the connector means is a hollow needle, the needle may be attached to or part of a media dispenser and serves as a connector means when it is injected directly into a container (e.g., through a rubber stopper covering a vial). In certain embodiments, the kit may comprise two or more inactive agent containers, for example, one container may be a base container and another container may be a non-base inactive container. In such a case, the non-base inactive container may be in the form of a media dispenser. The kit may further contain a mixing means, such as a mixing element. The mixing means may be contained in a container, incorporated into a container, or separate from any container. Non-limiting examples of mixing means for use in the present invention include: solid particles that aid in mixing the compounds (e.g., mixing balls) which are typically contained in the base container or may be in the kit separate from any container but then later added to the base container; and a mechanical stirrer which is typically incorporated into the base container or may be in the kit separate from any container but then later added to the base container. In an embodiment, the kit may comprise a recipient container into which the contents of the other containers (including, in some embodiments, the base container) may be transferred, for example by way of the media dispenser. The kit may also comprise a dispensing means which is associated with a base container, a recipient container, or is separate from any container. In the latter case, the dispensing means may be incorporated into a container or attached thereto prior to its use. Examples of such a dispensing means include a metered pump and a pump cap. The kit may additionally comprise a replacement base container, in which case the previously mentioned base container would be the primary base container. Each of the containers may individually be closed and airtight prior to their use. For example, a container which is a vial may be covered by a rubber stopper and a media dispenser may be covered with a cap. In an embodiment, the kit comprises a plurality of containers, for example two or more non-base inactive containers, two or more active agent containers, one primary base container, and a replacement base container. In an example of such an embodiment, each non-base inactive container may be associated with a specific active agent container and all active agent containers are associated with the single primary base container. In another such example, more than one non-base inactive container may be associated with a specific active agent container and all active agent containers are associated with the single primary base container. In yet another such example, a non-base inactive container may be associated with two or more active agent containers and all active agent containers are associated with the single primary base container.

In the practice of a method for making a compounded pharmaceutical product using the above kit, the inactive agent container is connected to an associated active agent container, for example, by a connector means such as Luer lock fittings. One of these containers is in the form of a media dispenser, for example a syringe. The media dispenser then is used to dispense the contents therein into the other container creating a mixture of the active agent and the inactive agent. The container containing the mixture may then be shaken or kneaded to further mix the contents therein. Alternatively, or in addition, the media dispenser may be used to mix the contents of the container by drawing the contents and then dispensing them back into the container. In an embodiment wherein the inactive agent is a solubilizer for the active agent, the container may be mixed until the active agent is fully solubilized. In an embodiment wherein the inactive agent is a suspending agent, the container may be mixed until a suspension of the active agent is achieved. The media dispenser is then used to draw either the full amount of the contents of the resulting mixture or a smaller metered amount thereof into the media dispenser. The media dispenser can then be used to dispense either the full amount of the contents thereof or a smaller metered amount. In embodiments wherein the kit further comprises a base or a recipient container, the media dispenser can be used to dispense either the full amount of the contents thereof or a smaller metered amount into said container. The aforementioned steps may be repeated or conducted in parallel if there are additional active agent containers and associated non-base inactive containers. In embodiments in which the media dispenser dispenses the contents thereof into a recipient container, the contents of a base container may also be dispensed into the recipient container. The kit may additionally comprise a replacement base container, in which case the previously mentioned base container would be the primary base container. In certain embodiments wherein a less than full metered amount of the active agent and non-base agent mixture is dispensed into the primary base container or a recipient container to which the base composition is added, the contents of a replacement base container may be added to said primary base container or recipient container to bring the amount of the compounded pharmaceutical up to a required amount. The kit may comprise a mixing means, in which case the mixing means may be used to mix the contents of the base container which now, in addition to the base composition, also contains the active agent(s) and the non-base inactive agent(s). For example, if the mixing means are solid particles that aid in mixing the compounds (e.g., mixing balls) contained in the base container, the container may be shaken to allow the balls to mix the contents therein. A dispensing means, either incorporated into the base container or added thereto is then used to dispense the resulting compounded pharmaceutical product.

In a particular embodiment, the kit as described above contains one vial containing levocetirizine, one vial containing cyanocobalamin, one vial containing mupirocin, a syringe containing deionized water, and two syringes containing benzyl alcohol. The vial containing levocetirizine is associated with the syringe containing deionized water and the vials containing cyanocobalamin and mupirocin are each associated with syringes containing benzyl alcohol. The kit may further comprise a base container, for example one containing a lotion base, and/or a mixing means. The kit may additionally comprise a replacement base container, in which case the previously mentioned base container would be the primary base container.

In another particular embodiment, the kit as described above contains one vial containing levocetirizine, one vial containing fluticasone, a syringe containing deionized water, a syringe containing benzyl alcohol, and a syringe containing sodium hydroxide. The vial containing levocetirizine is associated with the syringe containing deionized water and the vial containing fluticasone is associated with the syringe containing benzyl alcohol. The kit may further comprise a base container, for example one containing a lotion base, and/or a mixing means. The kit may additionally comprise a replacement base container, in which case the previously mentioned base container would be the primary base container.

In another particular embodiment, the kit as described above contains one vial containing tacrolimus monohydrate, one vial containing fluticasone propionate, and two syringes, each containing benzyl alcohol. Each of the vials is associated with one of the syringes. The kit may further comprise a base container, for example one containing a cream base, and/or a mixing means. The kit may additionally comprise a replacement base container, in which case the previously mentioned base container would be the primary base container.

In another embodiment of the present invention, the kit comprises at least one pouch which is a container or contains at least one container. In such an embodiment, the kit may also comprise at least one media dispenser which may serve as an active agent container, an inactive agent container, or a recipient container. In certain embodiments, the pouch contains at least two chambers and each of the chambers of the pouch is a container, as defined in the present specification. In a particular embodiment, at least one of such chambers is an active agent container and at least another such chamber is an inactive agent container (either a non-base inactive container or a base container). In such an embodiment, the kit may also comprise a media dispenser which may be an active agent container, an inactive agent container, or a recipient container. In another embodiment, all chambers within the pouch are active agent containers, in which case the kit may, for example, further comprise a media dispenser which may be an inactive agent container or the inactive agent container may be in a separate pouch with the media dispenser being a recipient container. In another embodiment, all chambers within the pouch are inactive agent containers, in which case the kit may, for example, further comprise media dispenser which may be an active agent container or the active agent may be in a separate pouch with the media dispenser being a recipient container. In the practice of the invention, the media dispenser may be attached to the pouch, for example by a connector means. In embodiments in which the media dispenser is an active agent container or an inactive agent container, the media dispenser can be used to mix the contents of the media dispenser with those of the pouch. In embodiments in which the media dispenser is a recipient container, the media dispenser can be used to extract the contents of a pouch. The contents may then be, for example, injected into another pouch or another chamber in the same pouch. In the event the second pouch or second chamber in the same pouch contains an active agent or an inactive agent, the media dispenser may be used to mix the contents contained therein with the contents of the second pouch or second chamber.

The containers within the pouch may be separated from each other by a common barrier (e.g., a wall, sheet, film, membrane, or a seal). The common barrier may be frangible or contain an element that is frangible, thus allowing the contents contained in the adjoining containers, following the fragmentation of the barrier or a frangible element therein, to mix with each other. It is contemplated that the pouch may, for example, be formed by two sheets (e.g., sheets of film) or one folded sheet which enclose(s) two or more chambers with the chambers separated from each other by areas wherein the two sheets, or two sides of a folded sheet, are sealed together to form an air-tight, but frangible seal. In embodiments wherein the pouch contains more than one frangible barrier, the barriers may be similarly fragmentable or may be more or less easily fragmentable relative to each other (e.g., fragmentable at a lower or higher pressure). The pouch may also contain a common barrier that is non-frangible. For example, in an embodiment, the pouch contains two pairs of associated containers, with each of the associated containers being separated from each other by a frangible common barrier or a common barrier containing a frangible element and the two pairs of containers being separated from each other by a non-frangible common barrier.

The kit may further contain at least one connector means, such as a connector element, for connecting the media dispenser to a container. The connector means may be incorporated into a container or may be separate but intended to be so incorporated. An example of such a connector means is a Luer lock. In certain embodiments, the pouch may contain a Luer lock fitting (e.g., a male or female Luer lock fitting) which allows for another container (e.g., a media dispenser) having the corresponding fitting to connect to the pouch. In such an embodiment, the Luer lock fitting may be contained on the pouch such that it connects to the interior of at least one of the internal chambers, thus allowing for the interior space of the chamber to connect with the interior space of the container with the corresponding Luer lock fitting. The pouch may also optionally further contain a frangible barrier between the chambers therein serving as the active agent and/or inactive agent containers and the chamber containing the connector means, for example, to prevent any of the contents of the active and/or inactive agent-containing chamber or chambers from discharging into or through the connector means. That frangible barrier may be similarly fragmentable as the frangible barrier(s) in between the chambers of the pouch or may be more or less easily fragmentable (e.g., fragmentable at a lower or higher pressure). For example, a pouch may contain a recipient container which contains or is accessible to the connector means and is separated from the active agent(s) and/or inactive agent(s) by the frangible barrier. As another example of a connector means, the kit may contain a hollow needle (e.g., a hypodermic needle) which may be part of attached to, or attachable to a media dispenser. In such embodiments, the hollow needle serves as a connector means when it is injected directly into a chamber of a pouch or injected into a fitting of the pouch that may be separated from a chamber of the pouch by a frangible barrier. In an example of a pouch which comprises a common barrier which is not frangible, chambers on either side of the barrier may each contain a connector means or be accessible to such a connector means (for example, by way through a frangible barrier between that chamber and another chamber which contains the connector means).

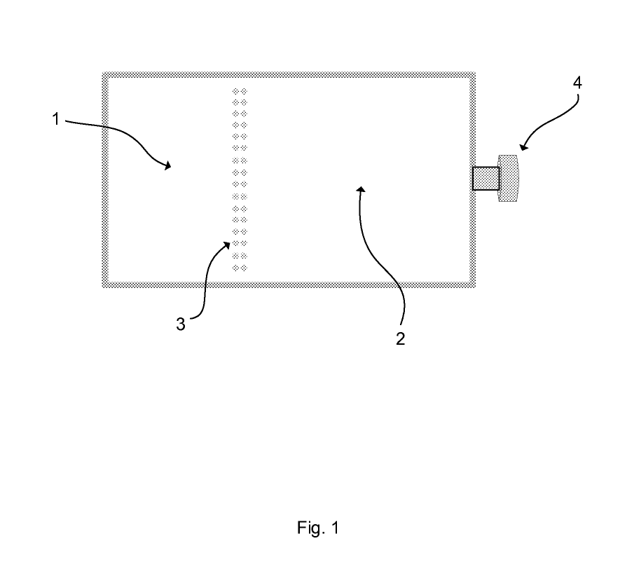

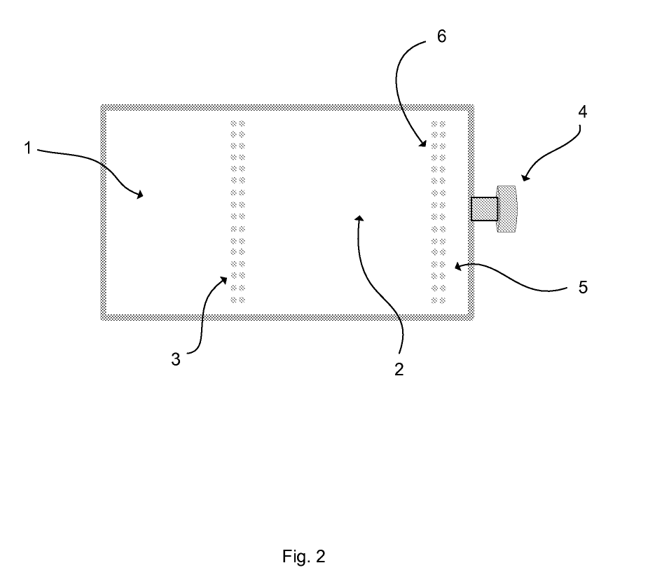

Embodiments of a pouch according to the present invention are illustrated by FIGS. 1 and 2 which serve as non-limiting examples of such pouches.

In FIG. 1, the pouch contains chambers 1 and 2. These chambers may each individually serve as an active agent container, an inactive agent container, or a recipient container. The pouch comprises a frangible barrier 3 in between the two chambers. The pouch also contains a connector means 4, such as a Luer Lock female fitting that allows for connection of the pouch to another container, such as a media dispenser (not shown).

FIG. 2 depicts another embodiment of the pouch wherein the pouch contains three chambers. Chambers 1 and 2 and the connector means 4 are as described above for FIG. 1. The pouch also contains another chamber 5 which serves as a recipient container and contains the connector means 4. That chamber 5 is separated from the other chambers 1 and 2 by a second frangible barrier 6 and is initially empty and serves as a buffer to prevent any unintended discharge of the active or inactive agents into or through the connector means until the time frangible barrier 6 is fragmented.

In an embodiment, the media dispenser has a volume that is greater than the combined volumes of all the chambers within said pouch. It is believed that such allows for all or nearly all of the contents of the pouch to be drawn into the media dispenser.

The kit may further comprise at least one recipient and/or base container that is separate and distinct from the previously described pouch(es) and media dispenser(s). The containers may be of any form, including vials, and pouches. The kit may further contain a mixing means, such as a mixing element. The mixing means may be contained in a container, incorporated into a container, or separate from any container. Examples of such a mixing means include mixing balls and/or a spatula, which may be present in the kit separately from any container but may later be add to the container to be used in the mixing of the contents thereof. The kit may also comprise a dispensing means which is incorporated into the base container or is separate from any container. In the latter case, the dispensing means may be incorporated into the base container or a recipient container or attached thereto prior to its use. Examples of such a dispensing means include a metered pump and a pump cap. Each of the containers may individually be closed and airtight prior to their use. For example, the chambers within a pouch may be sealed to be air-tight. In an embodiment, the pouch comprises three or more chambers which serve as containers, for example two or more non-base inactive containers and one active agent container or two or more active agent containers and one non-base inactive container.

The kit may contain a plurality of said pouches and said media dispensers, with each media dispenser being used to transfer the contents of a specific pouch to a base container or a recipient container. The contents of all the pouches may thus be transferred to a single base container, wherein they may be mixed. The kit may additionally comprise a replacement base container, in which case the previously mentioned base container would be the primary base container. If necessary, the contents of a replacement base container added may be added to the primary base container, to produce the compounded pharmaceutical product. In such embodiments, the replacement container may, for example, be added to achieve a desired weight and/or volume for the final compounded pharmaceutical product.

In the practice of a method for making a compounded pharmaceutical product using the above kit, pressure is applied to the pouch to fragment the barrier(s) between the chambers therein. This pressure may be applied by any means capable of fragmenting the barrier, such as by squeezing the pouch. In some embodiments, a cylindrical instrument such as a thin rod may be used by rubbing or rolling the instrument over the pouch to apply the necessary pressure to rupture the barrier. Following the fragmenting of the barrier(s) separating the chambers, the contents of the pouch may be mixed. Mixing may be done by any means, for example by hand or by kneading the pouch between the user's fingers and thumbs. A media dispenser may then be connected to the pouch, for example through a connector means already incorporated into the pouch (e.g., a Luer lock fitting) or added thereto. The media dispenser may be optionally used to mix the contents of the pouch by drawing the contents and then dispensing them back into the pouch. The media dispenser may then be used to draw or dispense either the full amount of the mixture or a smaller metered amount. In embodiments wherein the kit further contains a base container or a further recipient container, the media dispenser may be used to draw or dispense either the full amount of the mixture or a smaller metered amount into the container. The aforementioned steps may be repeated or conducted in parallel if there are additional pouches. In the embodiment in which the media dispenser dispenses the contents thereof into a recipient container, the contents of a base container may also be dispensed into the recipient container. The kit may additionally comprise a replacement base container, in which case the previously mentioned base container would be the primary base container. The kit may further contain a mixing means, such as a mixing element (e.g., a spatula or mixing balls). In embodiments in which a less than full metered amount of the active agent and non-base agent mixture is added to the primary base container or a recipient container to which the base composition is added, the contents of a replacement base container may be added to said primary base container or recipient container to bring the amount of the compounded pharmaceutical up to a required amount. In embodiments wherein the replacement base container is a media dispenser, the contents therein may be directly dispensed into the primary base container. In embodiments wherein the replacement base container is a pouch, a recipient container, for example in the form of a media dispenser, may be used to transfer the contents thereof into the primary base container. The contents of the primary base container may then be mixed using the mixing means, for example a spatula and/or mixing balls. The primary base container may contain a means for dispensing the contents thereof or such a means may then be added to the container. For example, a metered pump cap may be installed on top of the container. The primary base container may be in the form of a bottle.

In a particular embodiment, the kit as described above contains: (A) a pouch containing an active agent container containing prilocaine, a second active agent container containing lidocaine, and an inactive agent container containing a benzyl alcohol:propylene glycol solution as a solubilizer; and (B) a syringe for drawing the contents of the pouch. The kit may further comprise a base container, for example containing a gel base, and/or a mixing means, for example a spatula and/or mixing balls. The kit may additionally comprise a replacement base container, in which case the previously mentioned base container would be the primary base container.

In another particular embodiment, the kit as described above contains: (A) a pouch containing an active agent container containing benzocaine, and an inactive agent container containing propylene glycol; (B) a pouch containing one active agent container containing lidocaine, and an inactive agent container containing propylene glycol; and (C) two syringes, each for drawing the contents of a respective pouch. The kit may further comprise a base container, for example containing a gel base, and/or a mixing means, for example a spatula. The kit may additionally comprise a replacement base container, in which case the previously mentioned base container would be the primary base container.

In another particular embodiment, the kit as described above contains: (A) a pouch containing an active agent container containing tobramycin, and an inactive agent container containing purified water; (B) a pouch containing one active agent container containing phenytoin, and an inactive agent container containing a propylene glycol; (C) a pouch containing one active agent container containing lidocaine, and an inactive agent container containing propylene glycol; and (D) three syringes, each for drawing the contents of a respective pouch. The kit may further comprise a base container, for example containing a gel base, and/or a mixing means. The kit may additionally comprise a replacement base container, in which case the previously mentioned base container would be the primary base container.

In yet another particular embodiment, the kit as described above contains: (A) a pouch containing an active agent container containing collagenase, and an inactive agent container containing purified water; (B) a pouch containing one active agent container containing mupirocin, and an inactive agent container containing propylene glycol; and (C) two syringes, each for drawing the contents of a respective pouch. The kit may further comprise a base container, for example one containing an ointment base and/or a mixing means. The kit may additionally comprise a replacement base container, in which case the previously mentioned base container would be the primary base container.

In certain embodiments, the kit comprises a housing which is made of an air-tight impenetrable material and wherein voids within the housing form chambers which can serve as active agent containers, inactive agent containers, and/or recipient containers. The kit may also comprise a dispensing means which is capable of pumping or pushing the contents of the chambers within the housing simultaneously. The dispensing means may be part of the housing, be attachable to the housing, or be attached onto the housing. The kit may also comprise a mixing means. Any mixing means which can be effectively used may be used, including those previously described in this specification. In certain embodiments, the mixing means may be contained in a chamber which is part of the housing, is attached thereto, or is attachable thereto. Hereinafter in the specification and the claims, such a chamber will also be referred to as a “mixing chamber”. In certain embodiments, the dispensing means pumps or pushes the contents of the chambers within the housing simultaneously through a mixing chamber wherein the contents are mixed prior to being dispensed.

In a particular embodiment, one of the chambers of the aforementioned housing is a base container and another chamber is an active agent container, an inactive agent container, or a recipient container. It is noted that in such embodiments, while the recipient container is initially empty, in the practice of a method for making a compounded pharmaceutical product using the above kit, inactive agents and/or active agents may be added to the recipient container. For example, the kit may contain, in addition to the aforementioned housing, active agent containers, base containers, and non-base inactive containers outside of the housing. In a certain embodiment, the active agent(s) and non-base inactive agent(s) of the non-base inactive container may be combined and mixed by any means such as those previously described for other embodiments of the present invention. The resulting mixture may then be added to the recipient container of the housing.

It is contemplated that such kits may also comprise a replacement base container, in which case the previously mentioned base container would be the primary base container. In such embodiments, the contents of the replacement base container may be added to the primary base container or to the recipient container prior to dispensing.

Another aspect of the present invention is an apparatus for use in the production of a compounded pharmaceutical product. In an embodiment, the apparatus comprises a housing which contains at least two chambers, with each chamber serving as a container, a mixing chamber comprising a mixing means, and a dispensing means. In a certain embodiment, the apparatus comprises a mixing chamber through which the contents of the chambers are forced through when they are being dispensed using the dispensing means. In a certain embodiment of the apparatus, the active agent(s) and the inactive agent(s) are mixed at the time of dispersing. For example, a dispensing means may also comprise a mixing means (e.g., a static mixer).

In a further embodiment, the invention relates to a kit comprising the aforementioned apparatus.

In certain embodiments, the kit comprises a plurality of associated active agent containers and non-base inactive containers. Following the mixing of the active agents and non-base agents from associated containers, the mixtures from all the groups of the associated containers may be transferred to a recipient container in which they are further mixed. Then contents of this recipient container may then be transferred to a base container or to the recipient container that forms one of the chambers of the above-mentioned apparatus.

In a particular embodiment, the kit comprises a plurality of pouches which contain an active agent container and a non-base inactive container, a pouch that serves as a recipient container, and media dispensers. The contents of the pouches which contain the active agent container and non-base inactive container are mixed as described above and a media dispenser is used to transfer the mixture to the pouch that serves as a recipient container. Each of the active agent and non-base inactive agent mixtures are transferred into that pouch that serves as a recipient container. It may be that only a less than full metered amount is transferred. Then, if necessary, an amount of replacement base is also transferred into the recipient container from a replacement base container. The contents of the pouch that serves as a recipient container are mixed, for example by kneading the pouch. The contents of the pouch may then be added to a base container or to a further recipient container, for example, the recipient container that forms one of the chambers of the above-mentioned apparatus.

In embodiments for use in creating a compounded pharmaceutical product in the form of a suppository, the kit may, in addition to an active agent container and any inactive agent containers, also comprise a container which is heat resistant (e.g., a heat resistant jar). Such a jar, for example may be one that is able to withstand heat at a temperature of between about 40 and about 300° C., between about 60 and about 200° C., between about 60 and about 150° C., between about 60 and about 100° C., or between about 60 and about 80° C.

The heat resistant container may contain a base composition or may contain inactive agents which may be used to form a base composition. In the latter case, the kit may contain a further inactive container which also contains inactive agents which may be used to form the base composition and the contents of these inactive containers are mixed during the compounding process to form a base composition in situ.

In another embodiment, the heat resistant jar does not initially contain any inactive agents and the inactive agents are contained in separate inactive container(s). The other inactive containers may be a base container or inactive containers that contain inactive agents that may be later mixed to form a base. In such an embodiment, the inactive agents (either as a base composition or as separate inactive agents that form a base composition upon mixing) may later be transferred to the heat resistant container.

The heat resistant container may be heated until the contents therein are melted and then the active agent and any additional inactive agent (for example, inactive agents useful for forming a base composition in situ) may be added, for example by any of the methods described herein and using any of the implements described herein. The step of adding the active agent or mixture comprising an active agent to the base container may be repeated for any active agent or inactive agent. The contents of the container may then be mixed using the mixing means, for example a plastic stirrer, until uniform. The molten mixture may then be poured into a suppository mold, which may also be contained in the kit.

In embodiments in which a less than full metered amount of the active agent-containing mixture is added to a base container, the kit may additionally comprise a replacement base container. The contents of a replacement base container may be added to the heat resistant container to bring the amount of the compounded pharmaceutical up to a required amount. This may be accomplished, for example, using any of the methods described herein and using any of the implements described herein.

In a particular embodiment, the kit as described above contains: (A) a pouch containing an active agent container containing nifedipine, and an inactive agent container containing polyethylene glycol 300; (B) a pouch containing one active agent container containing lidocaine, and an inactive agent container containing polyethylene glycol 300; and (C) two media dispensers, each for drawing the contents of a respective pouch. The kit may further comprise a heat resistant container containing inactive agents therein appropriate for preparing a suppository, a mixing means, for example a plastic stirrer, and a suppository mold. The kit may also comprise a replacement base container.

In embodiments for use in creating a compounded pharmaceutical product in the form of a capsule, the kit may, in addition to an active agent container and any inactive agent containers, also comprise a triturating means, for example a mechanical means capable of grinding, milling or reducing the particle size of a powder mass or mixing a powder mass and a non-powder vehicle (e.g., a mortar and pestle).

In a particular embodiment, the kit as described above contains: (A) at least one sachet containing naltrexone hydrochloride; and (B) one base container in the form of a sachet containing lactose monohydrate. The kit may also comprise a triturating means such as a mortar and pestle. The kit may additionally comprise a replacement base container, in which case the previously mentioned base container would be the primary base container.

The compounded pharmaceutical product formed by the kits of the present invention may, for example, be in the form of a solid, liquid, gas, or semi-solid. Examples of the dosage form of the compounded pharmaceutical product include, but are not limited to: lotions, creams, gels, foams, ointments, solutions, suspensions, syrups, tablets, capsules, powders, granules, enemas, elixirs, parenterals, suppositories, and inhalants. The compounded pharmaceutical product may be administered by any appropriate means, for example, buccally, orally, parenterally, topically, transdermally, transmucosally, ophthalmically, otically, ocularly, periodontally, sublingually, intranasally, intramuscularly, rectally, vaginally, intraurethrally, subcutaneously, intravenously and by irrigational route.

In an embodiment, the kit is capable of producing multiple unit of use amounts of a compounded pharmaceutical product. A “unit of use amount” is the amount of the compounded pharmaceutical product needed to fill one prescription for one patient. In a particular such embodiment, the kit is capable of producing multiple unit of use amounts of said product with each unit having the same or different dosage strengths.

The kit of the present invention may also comprise instructions for the use thereof.

In a particular embodiment, the kit comprises all of the necessary elements for a user to form a compounded pharmaceutical product.

In an embodiment, the kit according to the present invention is one wherein said active agent(s) and said inactive agent(s) are mixed at the time of dispensing.

In an embodiment, the method for making the compounded pharmaceutical product is one wherein said active agent(s) and said inactive agent(s) are mixed at the time of dispensing.

Essentially any active agent capable of producing such a therapeutic effect in a subject to which it has been administered may be used in the practice of the present invention. Examples of such active agents in the context of the present invention include over-the-counter drug products, drug products available only by way of prescription, vitamin products, neutraceutical products and mineral supplements.

Examples of active agents for use in the present invention include, but are not limited to: 5-alpha reductase inhibitors, 5-aminosalicylates, abortifacients, acidifying agents, ADP receptor antagonists, adrenal agents, aldosterone antagonist, alkalinizing agents, alkylating agents, alpha blockers, alpha-glucosidase inhibitors, amino acids, aminoglycosides, amphenicols, amphetamines, amylin analogs, analgesics, androgens, anesthetics, angiotensin II receptor antagonist, angiotensin-converting enzyme inhibitor, anorectic agents, antacids, anthelmintics, anthracenediones, anti-anginals, anti-arrhythmics, antibiotics, anti-cholinergics, anti-convulsants, anti-diarrheals, anti-emetics, anti-estrogens, anti-fungal agents, anti-gout agents, anti-growth hormones, anti-histamines, anti-hypoglycemics, anti-infectives, anti-inflammatory agents, anti-manic agents, anti-metabolites, anti-migraine agents, anti-muscarinics, anti-mycobacterials, anti-neoplastic agents, anti-protozoals, anti-psoriatic agents, anti-spasmodics, anti-thyroid agents, anti-toxins, anti-tussive, anti-venins, anti-vertigo agents, anti-virals, aromatase inhibitors, atypical antipsychotics, autonomic agents, barbiturates, benzodiazepines, beta-blocker, biguanide, bile acid sequestrants, biologic response modifiers, bisphosphonates, bladder antiseptics, bone resorption inhibitors, calcium channel blocker, calcium modifiers, cannabinoid receptor antagonists, carbamates, carbapenems, carbonic anhydrase inhibitors, cardiac glycosides, cardiovascular agents, central-acting adrenergic agents, cephalosporins, chelating agents, chemotherapeutic agents, chemotherapy protectants, cholesterol absorption inhibitors, cholinesterase inhibitors, class IA antiarrhythmics, class IB antiarrhythmics, class IC antiarrhythmics, class II antiarrhythmics, class III antiarrhythmics, class IV antiarrhythmics, coagulation factors, colony stimulating factors, COMT inhibitors, contraceptives, corticosteroids, coumarin anticoagulants, cystic fibrosis transmembrane conductance regulators, decongestant, dermatological agents, dipeptidyl peptidase-4 inhibitor, direct renin inhibitor, disease-modifying antirheumatic drugs, electrolyte replacements, electrolytic and renal agents, emetics, enzymes, ergot alkaloids, estrogens, expectorants, fibric acid derivatives, fusion proteins, gadolinium-based contrast agents, gastric mucosal agents, gastrointestinal agents, gastrointestinal enzymes, genitourinary agents, glycopeptides, glycylcyclines, gonadotropin releasing hormone receptor antagonist, gonadotropin-releasing hormone (GnRH) analogs, H1 blockers, H2 blockers, heavy metal antagonists, hematinics, hematological agents, hemorrhoidal agents, heterocyclic antidepressants, HMG-CoA reductase inhibitor, hormones and hormone modifiers, hydantoins, hyperammonemia agents, hypotrichosis agents, immunoglobulins, immunosuppressants, incretin mimetics, inotropes, insulins, interferons, interleukins, ionic contrast media, keratinocyte growth factors, keratolytics, ketolides, laxatives, leukotriene receptor antagonists, lincosamides, lipopeptides, lipoxygenase inhibitors, long-acting respiratory beta-agonist, loop diuretics, low molecular weight heparins, macrolides, mast-cell stabilizers, meglitinides, metabolic agents, metabolic enzymes, methylxanthines, minerals, miotics, mixed opiate agonists/antagonists, monoamine depletors, monoamine oxidase inhibitors, monobactams, monoclonal antibodies, mucolytic agents, musculoskeletal agents, mydriatics, narcolepsy agents, neurological agents, neuromuscular blockers, nitroimidazoles, non-amphetamine stimulants, non-ionic contrast media, nonsteroidal anti-inflammatory drugs (NSAIDs), nutritional supplements, ophthalmic agents, opiate agonists, opiate antagonist, oropharyngeal agents, osmotic diuretics, otic agents, oxazolidinones, paramagnetic agents, parasympathomimetics, parathyroid agents, penicillins, peripheral mu-opioid receptor antagonist, phenotiazines, phosphate binding agents, phosphodiesterase inhibitors, photosensitizing agents, pigmentation agents, pituitary hormones, platelet glycoprotein IIb/IIIa inhibitors, polymyxins, polypeptides, porphyrins, potassium-sparing diuretics, progestins, prokinetic agents, prostaglandins, proton pump inhibitors, psychotropic agents, quinolones, radiopaue contract agents, radiopharmaceuticals, respiratory agents, respiratory enzymes, respiratory stimulating agents, retinoids, rifamycins, salicylates, scabicides, sclerosing agents, selective estrogen receptor modifiers, selective MAO-B inhibitors, selective norepinephrine reuptake inhibitors, selective serotonin reuptake inhibitors, serotonin-norepinephrine reuptake inhibitors, serotonin-receptor agonists, short-acting respiratory beta-agonist, signal transduction inhibitors, skeletal muscle relaxants, skin test antigens, sodium-glucose co-transporter 2 inhibitors, spermicides, sphingosine 1-phosphate receptor modulator, stool softeners, steroids, streptogins, succinimides, sulfonamides, sulfonylureas, sunscreens, surfactants, sympatholyrics, sympathomimetics, tetracyclines, therapeutic radiopharmaceuticals, thiazide diuretics, thiazolidinedione, thrombin inhibitors, thrombolytic agents, thrombopoietin receptor agonist, thyroid hormones, tocolytics, toxicology agents, toxoids, tricyclic antidepressants, tumor necrosis factor modifiers, uricosuric agents, vaccines, vasodilators, vasopressors, vitamin D analogs, vitamins, withdrawal agents and derivatives, analogs, enantiomers, isomers, complexes, salts, and mixtures thereof.

Examples of aminoglycosides which may be used in the present invention include, but are not limited to: amikacin, gentamicin, kanamycin, neomycin, paromomycin, streptomycin, tobramycin and derivatives, analogs, enantiomers, isomers, complexes, salts, and mixtures thereof.

Examples of anesthetics which may be used in the present invention include, but are not limited to: alfaxalone, ambucaine, amolanone, amoxecaine, amylocaine, aptocaine, articaine, benoxinate, benzocaine, benzyl alcohol, betoxycaine, biphenamine, bucricaine, bumecaine, bupivacaine, bupivicaine, butacaine, butamben, butanilicaine, carbizocaine, chloroprocaine, clibucaine, clodacaine, cocaine, dexivacaine, diamocaine, dibucaine, dyclonine, elucaine, etidocaine, etoxadrol, euprocin, fentanyl, fexicaine, fomocaine, heptacaine, hexylcaine, hydroxyprocaine, hydroxytetracaine, isobutamben, ketamine, ketocaine, leucinocaine, levoxadrol, lidocaine, mepivacaine, meprylcaine, methitural, methohexital, mexiletene, midazoiam, minaxolone, octocaine, orthocaine, oxethacaine, oxybuprocaine, parabutoxycaine, phenacaine, piperocaine, piridocaine, pinolcaine, polidocanol, pramocaine, pramoxine, prilocaine, propoxycaine, procaine, propanidid, propanocaine, proparacaine, propipocaine, propofol, propoxate, proxymetacaine, pyrrocaine, quatacaine, quinisocaine, remifentanyl, risocaine, rodocaine, ropivacaine, salicyl alcohol, sufentanyl, suicaine, tetracaine, tiletamine, trapencaine, trimecaine, vadocaine, zolamine, and derivatives, analogs, enantiomers, isomers, complexes, salts, and mixtures thereof.

Examples of anti-arrhythmic agents which may be used in the present invention include, but are not limited to: abanoquil, ACC-9164, acecainide, actisomide, adenosine, aimokalant, ajmaline, alinidine, alprafenone, amafolone, ambasilide, ameltolide, amiodaron, amiodarone, aprindine, artilide, asocainol, atenolol, AWD-G-256, azimilide, benderizine, benrixate, benzodioxine, bepridil, berlafenone, bertosamil, bidisomide, bisaramil, bisoprolol, BRL-32042, bucainide, bucromarone, bunaftine, bupivicaine, buquineran, butobendine, butoprozine, capobenic acid, carbizocaine, carcainium chloride, cariporide, carocainide, cercainide, cibenzoline, cifenline, ciprafamide, CL-284027, clamikalant, clofilium phosphate, CV-6402, CVT-510, cyclovirobuxine-D, D-230, detajmium bitartrate, dexsotalol, digoxin, diltiazem, dioxadilol, diprafenone, disobutamide, disopyramide, dofetilide, drobuline, dronedarone, droxicainide, E047/1, E-0747, E-4031, edifolone, emilium tosilate, emopamil, encainide, eproxindine, erocainide, ersentilide, esmolol, fepromide, flecainide, fluzoperide, gallanilide, glemanserin, guafecainol, GYKI-23107, GYKI-38233, heptacaine, hydroxyfenone, ibutilide, indecainide, ipazilide, itrocainide, ketocainol, L-702958, L-706000, levosemotiadil, lidocaine, lorcainide, lorajmine, magnesium sulfate, meobentine, metoprolol, mexiletine, milacainide, modecainide, moracizine, moxaprindine, murocainide, nibentan, nicainoprol, nipekalant, nofecainide, oxiramide, palatrigine, penticainide, pentisomide, phenytoin, pilsicainide, Pirmenol, pirolaz amide, prajmalium bitartrate, pranolium chloride, prifuroline, procainamide, propafenone, propranolol, pyrinoline, quinacainol, quindonium bromide, quinidine, recainam, rilozarone, risotilide, ronipamil, ropitoin, sematilide, sinomenine, solpecainol, sotalol, sparteine, SR-47063, stirocainide, stobadine, sulamserod, suricainide, tedisamil, terikalant, timolol, tiracizine, tocainide, tosifen, transcainide, trecetilide, verapamil, zocainone, and derivatives, analogs, enantiomers, isomers, complexes, salts, and mixtures thereof.

Examples of antibiotics which may be used in the present invention include, but are not limited to: ambisome, amikacin, aminoglycoside, amoxicillin, ampicillin, azithromycin, aztreonam, bacitracin, bactroban, blephamide, cefaclor, cefadroxil, cefdinir, cefepime, cefixime, cefoperazone, cefotaxime, cefotetan, cefoxitin, cefpodoxime, cefprozil, ceftazidime, ceftibuten, ceftizoxime, ceftriaxone, cephalexin, cephalosporin, cephazolin, cephradine, ceptaz, chloramphenicol, chloromycetin, chlorsig, ciprofloxacin, clarithromycin, clindagel, clindamycin, cloxacillin, colistin, cotrimoxazole, dalfoprictin, dapsone, daptomycin, dicloxacillin, doxycycline, duricef, ertapenem, erythromycin, floxin, gatifloxacin, gentamicin, imipenem, levaquin, levofloxacin, linezolid, loracarbef, macrolide, mefoxin, meronem, meropenem, metronidazole, minocycline, moxifloxacin, mupirocin, nafcillin, neomycin, nitrofurantoin, norfloxacin, ofloxacin, omnicef, penicillin, penicillin G, penicillin VK, piperacillin, pneumovax, polymixin, quincipristin, quinolone, rifampin, rifaximin, silver sulfadiazine, staphlex, streptomycin, sulfa trimethoprim, sulfacetamide, sulfonamides, targocid, tazarotene, telithromycin, tetracycline, ticarcillin, tigecycline, tinidazole, tobramycin, vancocin, zymar, and derivatives, analogs, enantiomers, isomers, complexes, salts, and mixtures thereof.

Examples of anti-convulsants which may be used in the present invention include, but are not limited to: albutoin, aloxidone, aminoglutethimide, atolide, beclamide, cinromide, citenamide, cyheptamide, dezinamide, dimethadione, eterobarb, ethadine, ethotoin, flurazepam, fluzinamide, ilepcimide, magnesium sulfate mephobarbital, methsuximide, milacemide, nabazenil, nafimidone, nimethazepam, nitrazepam, paramethadione, primidone, ralitoline, remacemide, ropizine, sabeluzole, stiripentol, sulthiame, thiopental sodium, tiletamine, gabapentin, phenytoin, phenobarbital, methylphenobarbital, clobazam, clonazepam, clorazepate, diazepam, midazolam, lorazepam, temazepam, nitrazepam, potassium bromide, felbamate, carbazempine, oxcarbazepine, valproic acid, sodium valproate, divalproex sodium, vigabatrin, progabide, tiagabine topiramate, pregablin, mephenytoin, phosphenytoin, trimethadione, primindone, levitiracetam, seletracetam, ethosuximide, phensuximide, mesuximide, acetazolamide, methazolamide, zonisamide, lamotrigine, phenacemide, pheneturide, valpromide, valnoctamide, perampanel, zoniclezole, and derivatives, analogs, enantiomers, isomers, complexes, salts, and mixtures thereof.

Examples of anti-fungal agents which may be used in the present invention include, but are not limited to: abafungin, albaconazole, amphotericin B, anidulafungin, bifonazole, buclosamide, butenafine, butoconazole, caspofungin, clotrimazole, econazole, fenticonazole, fluconazole, flucytosine, griseofulvin, isoconazole, itraconazole, ketoconazole, luliconazole, micafungin, miconazole, naftifine, nystatin, omoconazole, oxiconazole, sertaconzaole, sulconazole, terbinafine, terconazole, tiocoazole, tolciclate, tolindate, triacetin, undecylenic acid, voriconazole, and derivatives, analogs, enantiomers, isomers, complexes, salts, and derivatives, analogs, enantiomers, isomers, complexes, salts, and mixtures thereof.

Examples of anti-histamines which may be used in the present invention include, but are not limited to: antazoline, astemizole, azatadine, azelastine, barmastine, bromodiphenhydramine, brompheniramine, carbinoxamine, cetirizine, chlorcyclizine, chlorpheniramine, chlorpheniramine polistirex, cinnarizine, clemastine, closiramine, crivastine, cycliramine, cyclizine, cyproheptadine, dexbrompheniramnine, dexchlorpheniramine, dimethindene, diphenhydramnine, diphenidramine, dorastine, doxylamine, ebastine, fenethazine, fexofenadine, hidroxyzine, levocabastine, loratadine, medrilamyne, mianserin, noberastine, orphenadrine, pheniramine, promethazine, pyrilamine, pyrabrom, pyroxamnine, rocastine, rotoxamine, tazifylline, temelastine, terfenadine, tolpropamine, tripelennamine, triprolidine, zolamine and derivatives, analogs, enantiomers, isomers, complexes, salts, and derivatives, analogs, enantiomers, isomers, complexes, salts, and mixtures thereof.

Examples of anti-infectives which may be used in the present invention include, but are not limited to: ambisome, blephamide, mupirocin, tobramycin, amikacin, gentamicin, kanamycin, neomycin, paromomycin, paromycin, streptomycin, spectinomycin, rifaximim, ertapenem, dorpenem, imipenem, imipenem/cilastatin, meropenem, cefadroxil, cefazolin, cefalotin, cephalexin, cefaclor, cefamandole, cefoxitin, cefproxil, cefuroxime, cefixime, cefdinir, cefditoren, cefoperazone, cefotaxine, cefpodoxime, ceftazidime, cefibuten, ceftizoxime, ceftriaxone, cefepime, ceftaroline, ceftobiprole, vancomycin, telavancin, clindamycin, daptomycin, azithromycin, clarithromycin, erythromycin, telithromycin, spiramycin, aztreoname, nitrofurantoin, linezolid, loracarbef, amoxicillin, ampicillin, nafcillin, oxacillin, penicillin G, penicillin V, penicillin VK, piperacillin, tazobactam, clavulanate, sulbactam, colistin, polymyxin B, ciprofloxacin, enoxacin, gatifloxacin, levofloxacin, moxifloxacin, norfloxacin, ofloxacin, trovafloxacin, sparfloxacin, temafloxacin, sulfacetamide, sulfadizine, silver sulfadiazine, sulfadimethoxine, sulfamethizole, sulfamethoxazole, trimethoprim-sulfamethoxazole, doxycycline, minocycline, oxytetracycline, tetracycline, dapsone, capreomycine, cycloserine, ethambutol, ethionamide, isoniazid, pyrazinamide, rifampicin, rifabutin, rifapentine, streptomycin chloramphenicol, fosfomycin, metronidazole, tigecycline, tinidazole, trimethoprim, reptapamulin, bacitracin, chloromycetin, chlorsig, cloxacillin, cotrimoxazole, dalfoprictin, dicloxacillin, pneumovaxm, polymyxin, quinupristin, rifampin, rifaximin, tazarotene, ticarcillin, and derivatives, analogs, enantiomers, isomers, complexes, salts, and mixtures thereof.

Examples of anti-inflammatory agents which may be used in the present invention include, but are not limited to: aceclofenac, alclofenac, alclometasone, algestone, alpha amylase, amcinafal, amcinafide, amfenac, amiprilose, anakinra, anirolac, anitrazafen, apazone, balsalazide, bendazac, benoxaprofen, benzydamine, bromelains, bromfenac, broperamole, budesonide, carprofen, cicloprofen, cintazone, cliprofen, clobetasol, clobetasone, clopirac, cloticasone, cormethasone, cortodoxone, deflazacort, desonide, desoximetasone, dexamethasone, diclofenac, diflorasone, diflumidone, diflunisal, difluprednate, diftalone, dimethyl sulfoxide, drocinonide, endrysone, enlimomab, enolicam, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide, flunixin, meglumine, fluocortin butyl, fluorometholone, fluquazone, flurbiprofen, fluretofen, fluticasone, furaprofen, furobufen, halcinonide, halobetasol, halopredone, ibuprofen, ibufenac, ibuprofen, ilonidap, indoprofen, indomethacin, indoxole, intrazole, isoflupredone, isoxepac, isoxicam, ketoprofen, lofemizole, lonazolac, lornoxicam, loteprednol, meclofenamate, meclofenamic acid, meclorisone, mecoflenamic acid, mefenamic acid, mesalamine, meseclazone, methylprednisolone, momiflumate, nabumetone, naproxen, naproxol, nimazone, olsalazine, orgotein, orpanoxin, oxaprozin, oxyphenbutazone, paranyline, pentosan polysulfate, phenbutazone pirfenidone, piroxicam, piroxicam, piroxicam olamine, pirprofen, prednazate, prifelone, prodolic acid, proquazone, proxazole, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenoxicam, tesicam, tesimide, tetrydamine, tiopinac, tixocortol, tolfenamic acid, tolmetin, triclonide, triflumidate, zidometacin, and derivatives, analogs, enantiomers, isomers, complexes, salts, and mixtures thereof.

Examples of antimetabolites which may be used in the present invention include, but are not limited to: 6-azauracil, abacavir, adefovir, aminopterin, azacytidine, azathioprine, acyclovir, capecitabine, cladribine, clofarabine, cytosine arabinoside, cytarabine, cytarabine, cytarabine liposomal, didanosine, decitabine, doxifluridine, emtricitabine, entecavir, flexuridine, floxuridine, fludarabine, fluorouracil, forodesine, gemcitabine, gemcitabine hydrochloride, hydroxyurea, idoxuridine, lamivudine, mercaptopurine, methotrexate, nelarabine, pemetrexed, pentostatin, pralatrexate, pyrimethamine, raltitrexed, sapacitabine, stavudine, telbivudine, tenofovir, thiarabine, thioguanine, thiopurines, trifluridine, troxacitabine, vidarabine, zalcitabine, zidovudine, nand derivatives, analogs, enantiomers, isomers, complexes, salts, and mixtures thereof.

Examples of anti-neoplastic agents which may be used in the present invention include, but are not limited to: imiquimod, podophyllum, mechlorethamine, fluorourcil, alitretinoin, everolimus, eribulin mesylate, arsenic trioxide, busulfan, dacarbazine, altretamine, procarbazine, temozolomide, thiotepa, melphalan, cyclophosphamide, estramustine, ifosfamide, chlorambucil, bendamustine, carmustine, lomustine, streptozocin, carboplatin, oxaliplatin, cisplatin, mitoxantrone, hydroxyurea, pemetrexed, pralatrexate, methotrexate, nelarabine, cladribine, clofarabine, fludarabine, mercaptopurine, pentostatin, thioguanine, azacitidine, capecitabine, cytarabine, decitabine, gemcitabine, pomalidomide, lenalidomide, thalidomide, ipilimumab, sipuleucel-T, interferon-alfa-2a, interferon-alfa-2b, denileukin diftitox, romidepsin, aldeleukin, brentuximab, ofatumumab, bevacizumab, tositumomab, alemtuzumab, ramucirumab, cetuximab, obinutuzumab, gemtubumab ozogamicin, pertuzumab, rituximab, siltuximab, ibritumomab tiuxetan, bosutinib, vandetanib, cabozantinib, vismodegib, imatinib, trastuzumab, ponatinib, ibrutinib, axitinib, gefitinib, ruxolitinib, carfilzomib, sorafenib, dasatinib, sunitinib, erlotinib, nilotinib, lapatinib, vandetanib, panitumumab, bortezomib, pazopanib, crizotinib, ziv-aflibercept, vemurafenib, ceritinib, asparaginase, pegaspargase, omacetaxine, doxorubicin, daunorubicin, epirubicin, idarubicin, valrubicin, bleomycin, dactinomycin, plicamycin, mitomycin, irinotecan, topotecan, etoposide, teniposide, ixabepilone, paclitaxel, docetaxel, cabazitaxel, vincristine, vinorelbine, vinblastine, vinorelbine, methoxsalen, porfirmer, bexarotene, tretinoin, alitretinoin, I-131 tositumomab, radium Ra 223 dichloride, and derivatives, analogs, enantiomers, isomers, complexes, salts, and mixtures thereof.

Examples of anti-spasmodics which may be used in the present invention include, but are not limited to: alibendol, ambucetamide, aminopromazine, apoatropine, bevonium methyl sulfate, bietamiverine, butaverine, butropium bromide, carovehne, cimetropium bromide, cinnamedrine, clebopride, coniine hydrobromide, coniine, cyclobenzaprine, cyclonium, difemerine, diisopromine, dioxaphetyl, diponium, drofenine, emepronium, ethaverine, feclemine, fenalamide, fenoverine, fenpiprane, fenpiverinium, fentonium, flavoxate, flopropione, gluconic acid, guaiactamine, hydramitrazine, hymecromone, leiopyrrole, mebeverine, moxaverine, n,n-1trimethyl-3,3-diphenyl-propylamine, nafiverine, n-butylscopolammonium, octamylamine, octaverine, pentapiperide, phenamacide, phloroglucinol, pinaverium, piperilate, pip oxolan, pramiverin, prifinium, properidine, propivane, propyromazine, prozapine, racefemine, rociverine, spasmolytol, stilonium, sultroponium, tiemonium, tiquizium, tiropramide, t-mebutine, trepibutone, tricromyl, trifolium, tropenzile, trospium, xenytropium, and derivatives, analogs, enantiomers, isomers, complexes, salts, and mixtures thereof.

Examples of bile acid sequestrants which may be used in the present invention include, but are not limited to: cholestyramine, colestipol, colesevelam and derivatives, analogs, enantiomers, isomers, complexes, salts, and mixtures thereof.

Examples of calcium channel blockers which may be used in the present invention include, but are not limited to: amlodipine, bepridil, nifedipine, diltiazem, felodipine, isradipine, nicardipine, nisoldipine, verapamil and derivatives, analogs, enantiomers, isomers, complexes, salts, and mixtures thereof.

Examples of class IB antiarrhythmics which may be used in the present invention include, but are not limited to: mexiletine, lidocaine, tocainide and derivatives, analogs, enantiomers, isomers, complexes, salts, and mixtures thereof.

Examples of enzymes which may be used in the present invention include, but are not limited to: agalsidase beta, alglucerase, alpha-galactosidase enzyme, amylase, chymotrypsin, collagenase, dornase alpha, dehydrogenases, elastase, elosulfase alfa, endopeptidases, enolases, exopeptidase, hyaluronidase, hydrolases, idursulfase, imiglucerase, inulin, insulin, isomerases, kinases, lactase, lactobacillus species, laronidase, lipase, mutases, pancrelipase, pegademase, phospholipases, phosphorylases, proteases, proteinase, rasburicase, sapropterin, taliglucerase alfa, trypsin, velaglucerase, and derivatives, analogs, complexes, variants, and mixtures thereof.

Examples of H1 blockers which may be used in the present invention include, but are not limited to: azelastine, bepotastine, brompheniramine, carbinoxamine, cetirizine, chlorpheniramine, clemastine, cyproheptadine, desloratadine, dexchlorpheniramine, dimenhydrinate, diphenhydramine, doxylamine, fexofenadine, hydroxyzine, levocetirizine, loratadine, meclizine, naphazoline, olopatadine, promethazine, pyrilamine, triprolidine, and derivatives, analogs, enantiomers, isomers, complexes, salts, and mixtures thereof.

Examples of hydantoins which may be used in the present invention include, but are not limited to: ethotoin, fosphenytoin, phenytoin, and derivatives, analogs, enantiomers, isomers, complexes, salts, and mixtures thereof.

Examples of immunosuppressants which may be used in the present invention include, but are not limited to: anti-thymocyte immune globulin, ascomycin, azathioprine, cyclophosphamide, cyclosporine, daltroban, everolimus, gusperimus, mizoribine, mycophenolate, pimecrolimus, rapamycin, sirolimus, tacrolimus, vedolizumab, and derivatives, analogs, enantiomers, isomers, complexes, salts, and mixtures thereof.

Examples of keratolytics which may be used in the present invention include, but are not limited to: alpha hydroxy acids, azelaic acid, beta hydroxy acids, benzoyl peroxide, coal tar, cresols, dihydroxy benzene compounds, glycolic acid, isoretinoic acid, N-acetylcysteine, papain, phenol, podophyllum resin, pyuric acid, resorcinol, retinal, retinoids, retinoic acid, retinol, salicylic acid, selenium disulfide, sulfur, urea, vitamin A, and derivatives, analogs, enantiomers, isomers, complexes, salts, and mixtures thereof.

Examples of non-steroidal anti-inflammatory drugs which may be used in the present invention include, but are not limited to: aspirin, aceclofenac, alclofenac, alclometasone dipropionate, algestone acetonide, alpha amylase, amcinafal, amcinafide, amfenac sodium, amiprilose, anakinra, anirolac, anitrazafen, apazone, balsalazide disodium, bendazac, benoxaprofen, benzydamine, bromelains, bromfenac, broperamole, budesonide, carprofen, cicloprofen, choline salicylate, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, fenoprofen, flazalone, fluazacort, flufenamic acid, flumizole, flunisolide acetate, flunixin, flunixin, meglumine, fluocortin butyl, fluorometholone acetate, fluquazone, flurbiprofen, fluretofen, fluticasone, furaprofen, furobufen, halcinonide, halobetasol propionate, halopredone acetate, ibuprofen, ibufenac, ibuprofen aluminum, ibuprofen piconol, ilonidap, indoprofen, indomethacin, indomethacin sodium, indoxole, intrazole, isoflupredone acetate, isoxepac, isoxicam, ketoprofen, lofemizole, lonazolac, lornoxicam, loteprednol etabonate, magnesium salicylate, meclofenamate sodium, meclofenamic acid, meclorisone dibutyrate, mecoflenamic acid, mefenamic acid, meloxicam, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rofecoxib, rimexolone, romazarit, salcolex, salnacedin, salsalate, sanguinarium chloride, seclazone, sermetacin, sodium salicylate, sudoxicam, sulindac, suprofen, talmetacin, talniflumate, talosalate, tebufelone tenidap sodium, tenoxicam, tesicam, tesimide, tetrydamine, tiopinac, tixocortol pivalate, tolfenamic acid, tolmetin, tolmetin sodium, triclonide, triflumidate, valdecoxib, zidometacin, and derivatives, analogs, enantiomers, isomers, complexes, salts, and mixtures thereof.

Examples of opiate receptor antagonists which may be used in the present invention include, but are not limited to: methylnaltrexone, nalmafene, naloxone, naltrexone.

Examples of quinolones which may be used in the present invention include, but are not limited to: cinoxacin, ciprofloxacin, gatifloxacin, gemifloxacin, levofloxacin, lomefloxacin, moxifloxacin, nalidixic acid, norfloxacin, ofloxacin, sparfloxacin, trovafloxacin, and derivatives, analogs, enantiomers, isomers, complexes, salts, and mixtures thereof.

Examples of steroids which may be used in the present invention include, but are not limited to: alclometasone, beclomethasone, beclomethasone, betamethasone, budesonide, ciclesonide, clobetasol, corticosteroids, dehydroepiandrosterone (DHEA), desonide, dexamethasone, difulorasone, diflucortolone, estradiol, estriol, estrone, flucinonide, fludrocortisone, flunisolide, fluocinolone, fluocortolone, flurandrenolide, fluocinonide, fluticasone, halcinonide, halobetasol, hydrocortisone, methylprednisolone, mometasone, prednisolone, prednisolone, prednicarbate, prednisone, progesterone, testosterone, triamcinolone, and derivatives, analogs, enantiomers, isomers, complexes, salts, and derivatives, analogs, enantiomers, isomers, complexes, salts, and mixtures thereof.

Examples of skeletal muscle relaxants which may be used in the present invention include, but are not limited to: abobotulinum A, baclofen, chlorzoxazone, cyclobenzaprine, dantrolene, diazepam, incobotulinumtoxin A, metaxalone, onabotulinumtoxin A, ophenadrine, ophenadrine citrate, rimabotulinumtoxin B, and derivatives, analogs, enantiomers, isomers, complexes, salts, and mixtures thereof.

Examples of vitamins which may be used in the present invention include, but are not limited to: vitamin A, vitamin B, vitamin B1, vitamin B2, vitamin B5, vitamin B6, vitamin B12 (cyanocobalamin), vitamin C, vitamin D (calcitriol), vitamin D3, vitamin E, vitamin K1, biotin, calcipotriene, folic acid, and derivatives, analogs, enantiomers, isomers, complexes, salts, and mixtures thereof.

The active agent may be present in the kit in any amount. In an embodiment, the active agent is present in the kit in an amount capable of forming a single unit of use amount of a compounded pharmaceutical product. In another embodiment, the active agent is present in the kit in an amount capable of forming multiple unit of use amounts of a compounded pharmaceutical product, for example 1 to 100 units of use or 1 to 50 units of use.

The active agent is present in the compounded pharmaceutical product in a therapeutically-effective amount. A “therapeutically-effective amount” of an active agent is the amount of the active agent that is capable of producing a therapeutic response or desired effect in a subject to which the active agent has been administered. The amount that is considered therapeutically-effective may depend on the subject, for example, the age, condition, and gender of the subject. A therapeutically-effective amount will typically be from about 0.005 to about 99% by weight of the compounded pharmaceutical product, from about 0.005 to about 75%, from about 0.005 to about 50%, from about 0.005 to about 30%, from about 0.005 to about 15%, from about 0.005 to about 10%, from about 0.005 to about 7%, from about 0.005 to about 5%, or from about 0.05 to about 1% by weight of the compounded pharmaceutical product.

In certain embodiments, the active agent is present in an amount that is capable of forming a compounded pharmaceutical product with the active agent at its maximum desired dosage strength (e.g., at its highest desired concentration in the product). It is understood that it is not always desirable to administer an active agent to a patient at its maximum desired dosage strength. For example, it may be required that some patients (for example, children) be administered a lower dose (i.e., a compounded pharmaceutical product with a lower concentration of the active agent). However, the inclusion in the kit, which is capable of metering the active agent, of an amount of active agent that is capable of forming a compounded pharmaceutical product with the active agent at its maximum desired dosage strength allows for versatility on the part of the pharmacist or other user of the kit in that it allows for the user to add only the relevant or desired metered amount of the active agent to form the final compounded pharmaceutical product. For example, if it is desired that the patient be dosed at ½ the maximum dosage strength of an active agent, the full amount of the active agent(s) and the non-base inactive agent(s) may be mixed, but then only a smaller metered amount of the resulting mixture that contains the amount of active agent which forms a compounded pharmaceutical product having ½ the maximum desired dosage strength is added to the primary base container. In such cases, an amount of replacement base or non-base inactive may be added from the replacement base container to bring the final composition to its desired concentration. This may, for example, be accomplished by adding an amount of replacement base or non-base inactive to achieve a target weight amount for the final composition (for example, to equate the weight that the composition would have been at were it to contain the active agent(s) therein at full strength amounts) or to achieve a target volume amount for the final composition (for example, to equate the volume that the composition would have been at were it to contain the active agent(s) therein at full strength amounts).

The kit according to the present invention may also comprise at least one inactive agent. As previously discussed, the kit may comprise a base container which contains a base composition. Such base compositions serve to create the dosage form of the compounded pharmaceutical product. The kit may also comprise various non-base agents that are in addition to those contained in the base composition which are separately contained in a non-base inactive container.

The type of base composition used may depend on the dosage form desired. For example, for a compounded pharmaceutical product having the dosage form of a cream or lotion, a composition comprising an emulsifying agent as an inactive agent may be used. For a compounded pharmaceutical product having the dosage form of a gel, a composition comprising a gelling agent as an inactive agent may be used. For a compounded pharmaceutical product having the dosage form of a solution, a composition comprising a solvent as an inactive agent may be used. For a compounded pharmaceutical product having the dosage form of a suppository, inactive agents for use in such a composition may include a suspending agent, an oil, and/or any agent capable of maintaining a solid form at room temperature (approximately 25° C./77° F.) while maintaining a dissolved or suspended active agent in a homogeneous dispersion and being capable of being melted or significantly softened at body temperature. Such base compositions may also comprise essentially any other inactive agent, such as those which serve to form the desired dosage form or imparts properties to the dosage form. Examples of such additional inactive agents include, but are not limited to: water, oil, alcohols, solvents, surfactants, emulsifiers, thickeners, buffers, gelling agents, binders, disintegrants, suspending agents, dispersing agents, diluents, film forming agents, silicones, stabilizers, antimicrobial agents, solubilizers, adsorbants, humectants, penetration aids, lubricants, permeation enhancers, anti-oxidants, adsorbants, colorants, anti-foaming agents, foaming agents, tonicity agents, emollients, glidants, pH modifiers, bioadhesive chelating agents, flavoring agents, taste masking agents, sweeteners and any of the other inactive agents mentioned in the present specification and claims.

The base composition itself may also impart desirable properties. For example, it may serve as a medication. Examples of such base compositions include those which serve as a moisture barrier, a moisturizer, and/or an emollient.

The base composition may be obtained from a third party sources, such as from a commercial source. Examples of such base compositions include but are not limited to creams (e.g., Epicream®, Hylatopic®, Hylatoic Plus®, and Mimyx®), lotions, ointments, solutions, suspensions, shampoos, soaps, cleansers, powders, and gels.

The base composition may be present in any amount. In an embodiment, the base composition is present in an amount capable of forming the desired dosage form. For example, the base composition may be present in an amount of from about 1% to about 99.995%, from about 25% to about 99.995%, from about 50% to about 99.995%, from about 70% to about 99.995%, from about 85% to about 99.995%, from about 90 to about 99.995%, from about 93 to about 99.995%, from about 95 to about 99.995%, or from about 99 to about 99.995% by weight of the compounded pharmaceutical product.

In an embodiment, the inactive agent(s) is/are present in the kit in an amount capable of forming a single unit of use amount of a compounded pharmaceutical product. In another embodiment, the inactive agent(s) is/are present in the kit in an amount capable of forming multiple unit of use amounts of a compounded pharmaceutical product, for example 1 to 100 units of use or 1 to 50 units of use.

In embodiments of the present invention in which an emulsifying agent is used, essentially any suitable emulsifying agent or a mixture of two or more such emulsifying agents can be used. The resulting emulsions may be of any form, including, but not limited to: an oil-in-water emulsion, a water-in-oil emulsion, a water-in-oil-in-water emulsion, an oil-in-water-in-oil emulsion, a silicone in water in emulsion, a water in silicone emulsion, a silicone and oil in water emulsion, and a water in silicone and oil emulsion. The active agent of the compositions of the present invention may exist in either the continuous or the dispersed phase or in both phases depending upon whether the agent is hydrophilic, lipophilic, or amphiphilic.

Examples of emulsifying agents which may be used in the present invention include, but are not limited to: acacia, ammonium alginate, calcium alginate, caprylic/capric triglyceride, carbomer, calcium carboxymethylcellulose, carrageenan, cetyl alcohol, cholesterol, ethylene glycol stearate, glyceryl monostearate, glyceryl monooleate, cetostearyl alcohol, glyceryl behenate, hydroxypropyl cellulose, lanolin, lecithin, lauric acid, linoleic acid, stearic acid and stearyl alcohol, mysteric acid, myristyl alcohol, oleyl alcohol, octoduodecanol, palmitic acid and medium chain triglycerides, monoethanolamine, polyoxyethylene sorbitan fatty acid esters, polyoxyethylene stearate, polyoxyglycerides, sorbitan fatty acid esters, phospholipids, polyoxyehtylene fatty esters, vitamin E polyethylene glycol succinate, white wax, and monoethanolamine.

In embodiments of the present invention in which a gelling agent is used, essentially any suitable gelling agent or a mixture of two or more such gelling agents can be used. Gelling agents may be used to increase the viscosity of a compounded pharmaceutical.

Examples of gelling agents which may be used in the present invention include, but are not limited to: acacia, aluminum monostearate, gelatin, agar, alginic acid, sodium alginate, ammonium alginate, bentonite, calcium alginate, calcium silicate, polyvinyl pyrrolidone, potassium alginate, carbomers, carboxymethylcellulose sodium, carrageenan, microcrystalline cellulose and carboxymethylcellulose sodium, ceratonia, chitosan, corn starch, rice starch, maze starch, pregelatinzed starch, hydroxypropyl starch, ethyl cellulose, glyceryl behenate, glyceryl monooleate, glyceryl monostearate, glyceryl palmitostearate, guar gum, hydrophobic silicon dioxide, hydroxyethyl cellulose, hydroxyethylmethyl cellulose, hypromellose, magnesium aluminum silicate, methylcellulose, pectin, poloxamer, polycarbophil, polyethylene oxide, polymethacrylates, poly methyl vinyl ether maleic anhydride, polyoxyethylene alkyl ethers, hydrogenated castor oil, polyethoxylated castor oil, propylene carbonate, propylene glycol alginate, sodium hyaluronate, tragacanth, xanthan gum, zinc acetate, and mixtures thereof.

In embodiments of the present invention in which a solvent is used, essentially any suitable solvent or a mixture of two or more such solvents can be used. Examples of solvents which may be used in the present invention include, but are not limited to: water (e.g. purified water, deionized water, or “DI water”); and organic solvents such as alcohols (e.g. benzyl alcohol), ketones, esters, and alkanes.

In embodiments of the present invention in which a suppository is formed, essentially any suitable agent capable of forming a suppository can be used. Such agents can be classified as agents that are solid or agents that are liquid at room temperature (25° C./77° F.). Examples of agents that are solid at room temperature which may be used in forming suppositories include, but are not limited to: hydrogenated castor oil, cetostearyl alcohol, cetyl alcohol, cholesterol, gelatin, glyceryl behenate, glyceryl monooleate, glyceryl monostearate, glyceryl palmitostearate, palmitic acid, paraffin, stearic acid, stearyl alcohol, hydrogenated vegetable oil, anionic emulsifying wax, carnauba wax, cetyl esters wax, microcrystalline wax, nonionic emulsifying wax, white wax, yellow wax, and bees wax. Examples of agents that are liquid at room temperature which may be used in forming suppositories include, but are not limited to, almond oil, alpha tocopherol, vitamin E oil, canola oil, castor oil, coconut oil, corn oil, cottonseed oil, ethyl oleate, glycerin, mineral oil, octyldodecanol, oleic acid, oleyl alcohol, olive oil, peanut oil, petrolatum, propylene glycol, safflower oil, sesame oil, soybean oil, sunflower oil, and water. In addition to the above, water and agents soluble or miscible with water, oils and agents soluble or miscible with oils, emulsifiers, surfactants, silicones, antimicrobial agents, solvents, solubilizers, penetration aids, anti-oxidants, pH modifiers, chelating agents, anti-foaming agents, and any other inactive agents may be used in a suppository base composition.

In addition to the above, examples of additional inactive agents for use in the present invention include, but are not limited to: anti-foaming agents, buffering agents, acids, bases, foaming agents, organic acids, esters, alcohols, ketones, aldehydes, oils and agents soluble in or miscible with oil, salts, solvents, diluents, fillers, emulsifying agents, suspending agents, solubilizers, pH modifiers, surfactants, silicones, extracts, plasticizers, penetration aids, permeation enhancers, preservatives, carriers, emollients, vehicles, antioxidants, akalizing agents, acidifying agents, lubricants, antimicrobial agents, stabilizers, chelating agents, dispersing agents, viscosifiers, adsorbents, thickening agents, bioadhesives, humectants, gelling agents, flavoring agents, water and agents soluble in or miscible with water, and mixtures thereof. These agents may be part of the base composition or may be non-base agents.

Examples of anti-foaming agents which may be used in the present invention include, but are not limited to: dimethicone, hydrated silica gel, oleyl alcohol, polydimethylsiloxane, simethicone, and mixtures thereof.

Examples of buffering agents which may be used in the present invention include, but are not limited to: sodium bicarbonate, calcium carbonate, calcium formate, magnesium hydroxide, aluminum, aluminum hydroxide/magnesium hydroxide co-precipitate, aluminum hydroxide/sodium bicarbonate co-precipitate, calcium acetate, calcium bicarbonate, calcium borate, calcium bicarbonate, calcium citrate, calcium gluconate, calcium glycerophosphate, calcium hydroxide, calcium lactate, calcium phthalate, calcium phosphates, calcium succinate, calcium tartrate, calcium propionate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, and mixtures thereof.

Examples of plasticizers which may be used in the present invention include, but are not limited to: adipates, benzoates, alkyl citrates, phthalate-based plasticizers, sebacates, maleates, trimellitates, and mixtures thereof.

Examples of solubilizers which may be used in the present invention include, but are not limited to: alcohols, sodium taurocholate, caprylocaproyl polyoxyl-8 glycerides, polyethylene glycol, diethylene glycol monoethyl ether, propylene glycol, lauroyl polyoxyl-32 glycerides, polyoxyethylene hydrogenated castor oils, polysorbates, sorbitan esters, cyclodextrine including beta-cyclodextrin, sulfobutylether-beta-cyclodextrin, and 2 hydroxypropyl-beta-cyclodextrin, dimethylsulfoxide (DMSO), vitamin E polyethylene glycol succinate, alpha tocopherol, poloxamer, water, medium chain triglycerides, hypromellose acetate succinate, polyethylene glycol hydroxystearate, polyoxyl castor oil, polyoxyl hydrogenated castor oil, polyhydroxy stearate, polyoxyethylene stearate, polyoxy glyceride, pyrrolidone, triolein, and mixtures thereof.

Examples of alcohols for use as solubilizers include, but are not limited to, benzyl alcohols and ethanols. Water for use as a solubilizer may be, for example, purified water and deionized water (DI water).

Examples of suspending agents which may be used in the present invention include, but are not limited to: acacia, alginates, cellulose ethers, tragacanth, xanthan gum, bentonite, carbomers, carrageenan, gelatin, and mixtures thereof.

Examples of surfactants which may be used in the present invention include anionic, cationic, nonionic, zwitterionic, amphoteric and ampholytic surfactants, and mixtures thereof. Examples of surfactants which may be used in the present invention include, but are not limited to: cetrimide, cetylpyridium chloride, docusate sodium, lauric acid, phospholipids, pluronic acid, poloxamer, polyoxyethylene sorbitan fatty acid esters, polyoxyethylene stearate, polyoxyethylene hydrogenated castor oils, polyoxyglycerides, sodium lauryl sulfate, sorbitan fatty acid esters, vitamin E polyethylene glycol succinate, polyoxyehtylene fatty esters, and mixtures thereof.

Examples of thickening agents which may be used in the present invention include, but are not limited to: acacia, agar, ammonium alginate, beta-cyclodextrin, carboxymethylcellulose calcium, ceratonia, tragacanth gum, carboxymethylcellulose sodium, carrageenan, silicon dioxide, copovidone, povidone, ethylcellulose, methylcellulose, hypromellose, bentonite, polyvinyl alcohol, guar gum, hydroxyethyl cellulose, hydroxypropyl cellulose, sodium alginate, microcrystalline cellulose and carboxymethylcellulose sodium, xanthan gum, potassium alginate, polycarbofil, polydextrose, carbomer, carbomer copolymer, and mixtures thereof.

Examples of silicones which may be used in the present invention include, but are not limited to, silicone elastomers, dimethicone, chitosan, cyclomethicone, silicone copolymers, cyclopentasiloxane, cyclopentasiloxane and C30-C45 alkyl cetearyl dimethicone cross polymer, dimethicone and cetearyl dimethicone cross polymer, polysilicones, and mixtures thereof.

Examples of antimicrobial agents which may be used in the present invention include, but are not limited to, benzalkonium chloride, benzalthonium chloride, cetylpyridinium chloride, benzoic acid, benzyl alcohol, benzyl benzoate, boric acid, bronopol, butylene glycol, butyl paraben, cetrimide, chlorhexidine, chlorbutanol, chlorocresol, chloroxylenol, cresol, imidurea, phenol, propylparaben, methylparaben, sodium borate, sorbic acid, sodium metabisulfate, sodium sulfite, sulfur dioxide, and mixtures thereof.

Examples of stabilizers which may be used in the present invention include, but are not limited to: acacia, agar, alginic acid, ammonium alginate, carboxymethylcellulose calcium, carboxymethylcellulose sodium, carrageenan, microcrystalline cellulose and carboxymethylcellulose sodium, silicon dioxide, hypromellose, polycarbofil, polyoxyethylene stearate, polyvinyl alcohol, potassium alginate, povidone, sodium alginate, xanthan gum, and mixtures thereof.

Examples of solvents which may be used in the present invention include, but are not limited to: water, ethyl alcohol, propyl alcohol, almond oil, alpha tocopherol, ammonia solution, butylene glycol, coconut oil, corn oil, dimethyl sulfoxide, dimethylacetamide, ethyl oleate, oleic acid, olive oil, glycerin, isopropyl mysterate, isopropyl palmitate, mineral oil, peanut oil, phosphoric acid solution, sodium hydroxide solution, hydrochloric acid, potassium hydroxide solution, pyrrolidone, safflower oil, propylene glycol, propylene carbonate, sesame oil, soybean oil, including hydrogenated soybean oil, sulfuric acid solution, sunflower oil, and mixtures thereof.

Examples of penetration aids and permeation enhancers which may be used in the present invention include, but are not limited to: ethyl alcohol, glycofurol, propyl alcohol, alpha tocopherol, isopropyl mysterate, isopropyl palmitate, mysteric acid, myristyl acid, oleic acid, oleyl alcohol, palmitic acid, thymol, and triolein, vitamin E polyethylene glycol succinate, medium chain triglycerides, and mixtures thereof.

Examples of antioxidants which may be used in the present invention include, but are not limited to: alpha tocopherol, vitamin E oil, ascorbic acid, ascorbyl palmitate, butylated hydroxyanisole, butylated hydroxytolulene, edetic acid, erythorbic acid, propyl gallate, sodium ascorbate, citric acid, sodium thiosulfate, and mixtures thereof.

Examples of pH modifiers which may be used in the present invention include, but are not limited to: sodium hydroxide solution, hydrochloric acid, potassium hydroxide solution, sulfuric acid, triethanolamine, acetic acid, adipic acid, ascorbic acid, ammonia solution, boric acid, citric acid, fumaric acid, lactic acid, maleic acid, malic acid, potassium citrate, sodium citrate, sodium phosphate dibasic, sodium phosphate monobasic, propionic acid, sodium bicarbonate, sodium carbonate, sorbic acid, tartaric acid, and mixtures thereof.

Examples of chelating agents which may be used in the present invention include, but are not limited to, disodium edetate, edetic acid, citric acid, fumaric acid, malic acid, maltose, and pentetic acid, and mixtures thereof.

Examples of agents soluble in or miscible with water include, but are not limited to: polyethylene glycol, propylene glycol propylene carbonate, glycerin, polyoxyethylene sorbitan fatty acid esters, polyethylene glycol succinate, acacia, agar, ammonium alginate, tragacanth gum, carboxymethylcellulose sodium, carrageenan, copovidone, povidone, methylcellulose, hypromellose, polyvinyl alcohol, guar gum, hydroxyethyl cellulose, hydroxypropyl cellulose, sodium alginate, carboxymethylcellulose sodium, xanthan gum, potassium alginate, polycarbofil, polydextrose, carbomer, carbomer copolymer, cetylpyridinium chloride, boric acid, bronopol, cetrimide, chlorhexidine, chlorbutanol, chloroxylenol, imidurea, propylparaben, methylparaben, sodium borate, sorbic acid, sodium metabisulfate, sodium sulfite, sulfur dioxide, sodium hydroxide solution, hydrochloric acid, potassium hydroxide solution, sulfuric acid, triethanolamine, acetic acid, adipic acid, ascorbic acid, ammonia solution, boric acid, citric acid, fumaric acid, lactic acid, maleic acid, malic acid, potassium citrate, sodium citrate, sodium phosphate dibasic, sodium phosphate monobasic, propionic acid, sodium bicarbonate, sodium carbonate, sorbic acid, and tartaric acid, disodium edetate, edetic acid, citric acid, fumaric acid, malic acid, maltose, pentetic acid, and mixtures thereof.

Examples of oils or materials that are soluble or miscible with oils include, but are not limited to: Vitamin E oil, alpha tocopherol, mineral oil, coconut oil, corn oil, cottonseed oil, castor oil, isopropyl mysterate, isopropyl palmitate, lanolin, linoleic acid, oleic acid, peanut oil, vegetable oil, benzyl alcohol, paraffin, white wax, microcrystalline wax and medium chain triglycerides, ethylcellulose, benzyl alcohol, benzoic acid, benzyl benzoate, butylene glycol, butyl paraben, chlorocresol, cresol, phenol, cetyl alcohol, cholesterol, glyceryl monostearate, cetostearyl alcohol, glyceryl behenate, lauric acid, linoleic acid, stearic acid and stearyl alcohol, mysteric acid, myristyl alcohol, oleyl alcohol, octoduodecanol, palmitic acid, medium chain triglycerides, and mixtures thereof.

Examples of foaming agents which may be used in the present invention include, but are not limited to: fatty alcohols, sodium laureth sulfate, sodium lauryl ether sulfate, sodium lauryl sulfate (also known as sodium dodecyl sulfate), triethanolamine lauryl sulfate, ammonium lauryl sulfate (ALS), and mixtures thereof.

Examples of sweeteners which may be used in the present invention include, but are not limited to: fructose, glucose, sucrose, saccharin, aspartame, acesuflame potassium, mannitol, sucralose, sorbitol, xylitol, and mixtures thereof.

Examples of preservatives which may be used in the present invention include, but are not limited to: butylated hydroxy toluene, butylated hydroxy anisole, methyl parahydroxybenzoate, propyl parahydroxybenzoate and sodium benzoate and mixtures thereof.

Examples of flavoring agents which may be used in the present invention include, but are not limited to: cherry, vanilla, strawberry, lemon, yoghurt, cardamom, fennel, peppermint, anise, and mixtures thereof.

The non-base inactive agent may be present in any amount. In an embodiment, the non-base inactive agent is present in an amount capable of imparting the desired properties onto the compounded pharmaceutical. For example, the non-base inactive agent may be present in an amount up to about 95%, up to about 50%, up to about 25%, up to about 10%, up to about 5%, or up to about 1% by weight of the compounded pharmaceutical product.

In an embodiment of the present invention, the compounded pharmaceutical product is one wherein the individual assay values of samples taken therefrom range from about 60% to about 140% of the label claim for the drug substance, from about 70% to about 130% of the label claim for the drug substance, from about 80% to about 120% of the label claim for the drug substance, from about 90% to about 110% of the label claim for the drug substance, or from about 95% to about 100% of the label claim for the drug substance.

In an embodiment of the present invention, kits of the same design and composition are capable of producing multiple compounded pharmaceutical products having the same or similar active agent and/or inactive agent content, in terms of strength (average weight percentage) and content uniformity (individual dose uniformity).

In another embodiment of the present invention, the kit is capable of producing multiple unit of use amounts of the compounded pharmaceutical product, each having the same or similar active agent and/or inactive agent content, in terms of strength (average weight percentage) and content uniformity (individual dose uniformity).

In preferred embodiments, the product has an Acceptance Value (AV), as defined in the United States Pharmacopeia, of less than 15 when 10 units thereof are tested, less than 10 when 10 units are tested, less than 5 when 10 units are tested, or less than 1 when less than 10 units are tested. In another embodiment, the product has an AV of less than 25, less than 20, less than 15, less than 10, less than 5, or less than 1, when 30 units are tested.

In an embodiment, the assay values of the amounts of an active and/or inactive agents present in the products, unit of use amounts, or samples to be compared have a relative standard deviation (% RSD) of less than about 20%, less than about 15%, less than about 10%, less than about 8%, less than about 6%, less than about 5%, less than about 3%, less than about 2%, less than about 1%, less than about 0.5%, or less than about 0.1%.

In an embodiment of the present invention, the kit is capable of producing a stable compounded pharmaceutical product. The stability of a product, in the present context, refers to the chemical and physical integrity of the product and, when appropriate, the ability of the product to maintain protection against microbiological contamination.

The stability parameters of a drug dosage form can be influenced by environmental conditions of storage (temperature, light, air, and humidity), as well as the package components.

Examples of compounded pharmaceutical products of the present invention include, but are not limited to, combinations of the following active agents: lidocaine and prilocaine; tracrolimus and fluticasone; tobramycin and phenytoin; nifedipine and lidocaine; benzocaine and lidocaine; tobramycin, phenytoin, and lidocaine; diclofenac, lidocaine, and prilocaine; levofloxicin, tobramycin, and nystatin; collagenase and hyaluronidase; mupirocin, levocetirizine, and cyanocobalamin; collagenase and mupirocin; calcipotriene, fluticasone, and tacrolimus or sirolimus; levocetirizine and fluticasone; ketamine, gabapentin, diclofenac, and lidocaine or bupivacaine; fluorouracil and salicylic acid; nifedipine and lidocaine; and itraconazole and undecylenic acid.

Specific examples of a compounded pharmaceutical product of the present invention include, but are not limited to: a dermatological lotion comprising levocetirizine dihydrochloride, cyanocobalamin, and mupirocin; a dematological lotion comprising levocetirizine dihydrochloride and fluticasone propionate; a pain management gel comprising lidocaine and prilocaine; a dermatological cream comprising tacrolimus monohydrate and fluticasone proprionate; a wound care ointment comprising collagenase and mupirocin; a wound care gel comprising tobramycin, lidocaine hydrochloride, and phenytoin; a suppository comprising nifedipine and lidocaine for use in controlling anal fissures; a capsule comprising naltrexone hydrochloride for pain management; and a gel comprising benzocaine and lidocaine for use as a topical anesthetic.

### EXAMPLES

### Example 1

### Preparation of a Dermatological Cream Base

250 g of a cream base was prepared according to the formula and process outlined below.

An 18% sodium hydroxide stock solution was prepared as follows. 410 g of purified water was weighed into an appropriately-sized stainless steel beaker. A magnetic stir bar was added to the beaker and the beaker was placed on a hot/stir plate with the heat off. Stirring was initiated at a speed sufficient to create a vortex. 90 g of sodium hydroxide (distributed by Professional Compounding Centers of America) was added slowly and allowed to dissolve with mixing for 30 minutes.

A water and Carbomer Interpolymer Type A mixture was prepared as follows. 20.83 g of purified water was weighed into an appropriately-sized stainless steel beaker. A magnetic stir bar was added to the beaker and the beaker was placed on at hot/stir plate with the heat off. Stirring was initiated at a speed sufficient to create a vortex. 0.63 g of Carbomer Interpolymer Type A (marketed by Lubrizol as Carbopol® Ultrez 10 NF) was added slowly to the purified water and mixed for 10 minutes.

A caprylic/capric triglyceride and Carbomer Copolymer Type A mixture was prepared as follows. 10 g of caprylic/capric triglyceride (marketed by Abitec Corp. as Captex® 355 NF) was weighed into an appropriately sized stainless steel beaker. A magnetic stir bar was added to the beaker and the beaker was placed on at heat/stir plate with the heat off. Stirring was initiated at a speed sufficient to create a vortex. 0.75 g of Carbomer Copolymer Type A (marketed by Lubrizol as Pemulen™ TR-2 NF) was added slowly to the caprylic/capric triglyceride with and mixed for 10 minutes.

86.66 g of purified water was weighed into an appropriately-sized stainless steel beaker. A magnetic stir bar was added to the beaker and the beaker was placed on at hot/stir plate with the heat off. Heating and stirring was initiated at a speed sufficient to create a vortex. Once the temperature of the mixture reached 60° C., 0.25 g of benzoic acid (distributed by PCCA Inc.) was added and the mixture was allowed to continue to heat until the temperature was 65-70° C. This mixture was referred to as the water phase.

62.5 g of silicone elastomer, cyclopentasiloxane and polysilicone-11 gel (marketed by Grant Industries as Gransil RPS) was weighed into an appropriately sized stainless steel beaker. The beaker was placed on a hot plate. Mixing, using a Lightnin LabMaster G3U05R (SPX Corporation) overhead mixer, and heating was initiated. The following ingredients were added to the silicone elastomer with continued heating and mixing: 15 g of dimethicone and cetearyl dimethicone copolymer (marketed by Momentive Performance Materials Inc. as Velvesil DM); 17.5 g of glyceryl monostearate (marketed by BASF Corp. as Kolliwax GMS II); 0.75 g of polysorbate 80 (marketed by Croda as Tween 80); 0.38 g of sorbitan monooleate (marketed by Croda as Span 80); 3 g of vitamin E oil (marketed by Natural Sourcing); 8.75 g of pracaxi oil (marketed by Natural Sourcing); 0.25 g of butylated hydroxytolulene (marketed by Merck); 7.5 g of polyethylene glycol-16 macadamia glycerides, (marketed by Floratech as Florasolvs PEG-16 macadamia glycerides); 10 g of isopropyl myristate (distributed by Professional Compounding Centers of America); all of the water and Carbomer Interpolymer Type A mixture prepared above; and all of the caprylic/capric triglyceride and Carbomer Copolymer Type A mixture prepared above. The mixture was mixed with a Lightnin Labmaster G3U05R (SPX Corporation) overhead mixer with continued heating. Once the temperature reached 55° C., 3.25 g of phosphatidylcholine (marketed by Lipoid as Phosphlipon® 90G) was added and the mixture was allowed to continue to heat. The mixture was mixed with overhead stirring until the temperature reached 65-70° C. The mixture was referred to as the oil phase.

When both the water phase and the oil phase reached a temperature of 65-70° C., the oil phase was slowly added to the water phase with continued mixing using a Lightnin Labmaster G3U05R (SPX Corporation) overhead mixer. The heat was turned off and the mixture was allowed to slowly cool. Once the mixture cooled to 35° C., 2 g of the 18% sodium hydroxide solution was added slowly with continued mixing. The mixture which formed an emulsion was cooled to room temperature. The pH was measured and determined to be 5.4. The cream base emulsion was packaged in a jar with a screw top lid.

### Example 2

### Preparation of a Dermatological Lotion at Maximum Strength for all Active Ingredients Including Levocetirizine Dihydrochloride, Cyanocobalamin and Mupirocin

### Preparation of Primary Base Container

A lotion base was prepared from the cream base, prepared according to the formula and process outlined in Example 1, by mixing 66.6 g of the cream base with 30 g of water, 0.7 g of xanthan gum (marketed by CP Kelco Co. as Xantural® 75), and 2.7 g of 18% sodium hydroxide solution.

87.93 g of the lotion base was filled into a pump bottle. Three 3.5 mm stainless steel balls were added as non-removable mixing aids.

### Preparation of Active Agent Containers

A fixed amount of levocetirizine dihydrochloride (distributed by Professional Compounding Centers of America), equivalent to the maximum desired strength (2 g or 2% of the final product), was filled into a vial. A cap with a rubber stopper were applied to the vial with a crimper. The vial was appropriately labelled as “Levocetirizine Dihydrochloride 2 g/2%”.

A fixed amount of cyanocobalamin (distributed by Professional Compounding Centers of America), equivalent to the maximum desired strength (0.07 g or 0.07% of the final product), was filled into a vial. A cap with a rubber stopper were applied to the vial with a crimper. The vial was appropriately labelled as “Cyanocobalamin 0.07 g/0.07%”.

A fixed amount of mupirocin (distributed by Professional Compounding Centers of America), equivalent to the maximum desired strength (2 g or 2% of the final product), was filled into a vial. A cap with a rubber stopper were applied to the vial with a crimper. The vial was appropriately labelled as “Mupirocin 2 g/2%”.

### Preparation of Non-Base Inactive Containers

A fixed amount of purified water, in an amount sufficient to solubilize the levocetirizine dihydrochloride, (3 g or 3% of the final product) was filled into a syringe (manufactured by Becton, Dickinson and Company) with a Luer lock fitting. A cap was then applied to the syringe. The syringe was appropriately labelled as “Purified Water 3 g/3% for Levocetirizine Dihydrochloride”.

A fixed amount of benzyl alcohol (distributed by Professional Compounding Centers of America), in an amount sufficient to solubilize the cyanocobalamin, (1 g or 1% of the final product) was filled into a syringe (manufactured by Becton, Dickinson and Company) with a Luer lock fitting. A cap was then applied to the syringe. The syringe was appropriately labelled as “Benzyl Alcohol 1 g/1% for Cyanocobalamin”.

A fixed amount of benzyl alcohol, in an amount sufficient to solubilize the mupirocin, (4 g or 4% of the final product) was filled into a syringe (manufactured by Becton, Dickinson and Company). A cap was then applied to the syringe. The syringe was appropriately labelled as “Benzyl Alcohol 4 g/4% for Mupirocin”.

### Preparation of Replacement Base Container

A fixed amount of the aforementioned lotion base, in an amount sufficient to replace all of the active ingredients and active solubilizing fluids (12.07 g or 12.07% of the final product), was filled into a syringe (manufactured by Becton, Dickinson and Company) with a Luer lock fitting. A cap was then applied to the syringe. The syringe was appropriately labelled as “Replacement Lotion Base 12.07 g/12.07% for qs”.

### Mixing of Active Agents, Non-Base Inactive Agents, and Base Composition

A needleless vial adapter (marketed by Yukon Medical as a 20 mm Vented ViaLok®), was installed onto the vial labelled “Levocetirizine Dihydrochloride 2 g/2%”. The syringe labelled “Purified Water 3 g/3% for Levocetirizine Dihydrochloride” was installed onto the needleless vial adapter. The purified water in the syringe was injected into the vial and the contents of the vial were mixed by shaking the vial and by using the syringe to draw and dispense the contents until all of the levocetirizine dihydrochloride was solubilized. The levocetirizine dihydrochloride solution was then drawn from the vial using the syringe. The entire quantity of solution was injected into the open pump bottle containing the lotion base and three (3) 3.5 mm stainless steel balls.

A needleless vial adapter (marketed by Yukon Medical as a 20 mm Vented ViaLok®), was installed onto the vial labelled “Cyanocobalamin 0.07 g/0.07%”. The syringe labelled “Benzyl Alcohol 1 g/1% for Cyanocobalamin” was installed onto the needleless vial adapter. The benzyl alcohol was injected into the vial and the contents of the vial were mixed by shaking the vial and by using the syringe to draw and dispense the contents until all of the cyanocobalamin was solubilized. The cyanocobalamin solution was then drawn from the vial using the syringe. The entire quantity of solution was injected into the open pump bottle containing the lotion base and three (3) 3.5 mm stainless steel balls.

A needleless vial adapter (marketed by Yukon Medical as a 20 mm Vented ViaLok®0029, was installed onto the vial labelled mupirocin 2 g/2%. The syringe labelled “Benzyl Alcohol 4 g/4% for Mupirocin” was installed onto the needleless vial adapter. The benzyl alcohol was injected into the vial and the contents of the vial were mixed by shaking the vial and by using the syringe to draw and dispense the contents until all of the all of the mupirocin was solubilized. The mupirocin solution was then drawn from the vial using the syringe. The entire quantity of solution was injected into the open pump bottle containing the lotion base and three (3) 3.5 mm stainless steel balls.

A pump cap was installed onto the bottle containing the lotion base and solubilized active ingredients and tightened to seal the bottle. The bottle was shaken continuously for one (1) minute while tapping the bottom of the bottle firmly on the palm of the hand.

The content uniformity of the lotion was determined by assaying product from ten individual pumps spaced equally over the content of the bottle. After the pump was primed, product from ten pumps was collected by taking product from approximately every tenth pump. The product from each pump was weighed and assayed for content of each drug. Table 1 below shows the weight and assay results for each active ingredient.

TABLE 1

| | | Cyanocobalamin | | Mupirocin | | Levocitirizine dihydrochloride | |
|---|---|---|---|---|---|---|---|
| Sample | Pump Sample | | % Label | | % Label | | % Label |
| No. | Wt. (g) | Wt. (mg/g) | Claim¹ | Wt. (mg/g) | Claim² | Wt. (mg/g) | Claim³ |
| 1 | 1.008 | 0.684 | 97.71 | 19.14 | 95.70 | 19.36 | 96.80 |
| 2 | 0.996 | 0.699 | 99.86 | 18.58 | 92.90 | 19.16 | 95.80 |
| 3 | 1.014 | 0.682 | 97.43 | 19.69 | 98.45 | 19.35 | 96.75 |
| 4 | 1.003 | 0.696 | 99.43 | 19.40 | 97.00 | 19.55 | 97.75 |
| 5 | 1.000 | 0.723 | 103.29 | 19.92 | 99.60 | 19.43 | 97.15 |
| 6 | 1.009 | 0.711 | 101.57 | 19.20 | 96.00 | 19.32 | 96.00 |
| 7 | 0.999 | 0.694 | 99.14 | 19.20 | 96.00 | 20.42 | 102.10 |
| 8 | 1.000 | 0.696 | 99.43 | 19.22 | 96.10 | 20.78 | 103.90 |
| 9 | 0.998 | 0.682 | 97.43 | 19.17 | 95.85 | 19.98 | 99.90 |
| 10 | 1.009 | 0.701 | 100.14 | 19.71 | 98.55 | 20.22 | 101.10 |
| Average | 1.004 ± 0.006 | 0.697 ± 0.013 | 99.54 ± 1.86 | 19.32 ± 0.38 | 96.62 ± 1.90 | 19.76 ± 0.55 | 98.79 ± 2.77 |
| Standard | 0.006 | 0.013 | 1.86 | 0.38 | 1.90 | 0.55 | 2.77 |
| Deviation | | | | | | | |
| USP | | | 4.45 | | 6.44 | | 6.66 |
| Acceptance | | | | | | | |
| Value (AV) | | | | | | | |

¹Label Claim: 0.7 mg/g (0.07%)
²Label Claim: 20 mg/g (2.0%)
³Label Claim: 20 mg/g (2.0%)

The weight of formulation per pump was 1.004 g with a standard deviation of 0.006 g and a maximum single pump deviation from the target weight of 1.000 g (for a single pump) of 0.014 g.

The average amount of cyanocobalamin contained in a gram of lotion product was 0.697 mg or 0.07% w/w. The theoretical amount was 0.07 mg/g or 0.07%. The relative standard deviation was 1.87% (0.013/0.697*100). The values were well within traditionally established and current USP acceptance criteria for semi-solid products. The assay values were within 90-110% of label claim (0.063-0.077%) with a relative standard deviation of less than or equal to 6% and the Acceptance Value (AV) was 4.45 (below the limit of 15).

The average amount of mupirocin contained in a gram of lotion product was 19.32 mg or 1.93% w/w. The theoretical amount was 20.0 mg/g or 2% w/w. The relative standard deviation was 1.97% (0.38/19.32*100). The values were well within traditionally established and current USP acceptance criteria for semi-solid products. The assay values were within 90-110% of label claim (1.80-2.20%) with a relative standard deviation of less than or equal to 6% and the Acceptance Value (AV) was 6.44 (below the limit of 15).

The average amount of levocetirizine dihydrochloride contained in a gram of lotion product was 19.76 mg or 1.97% w/w. The theoretical amount was 20.0 mg/g or 2% w/w. The relative standard deviation was 2.78% (0.55/19.76*100). The values were well within traditionally established and current USP acceptance criteria for semi-solid products. The assay values were within 90-110% of label claim (1.80-2.20%) with a relative standard deviation of less than or equal to 6% and the Acceptance Value (AV) was 6.66 (below the limit of 15).

### Example 3

### Preparation of a Dermatological Lotion at Half Strength for all Active Ingredients Including Levocetirizine Dihydrochloride, Cyanocobalamin and Mupirocin

The primary base container, the active agent containers, the non-base inactive containers, and the replacement base container were prepared as described above for Example 2. Additionally each of the active agents (levocetirizine dihydrochloride, cyanocobalamin and mupirocin) was mixed with its respective non-base inactive agent as described in Example 2.

2.3 mL of levocetirizine dihydrochloride solution (containing 1 g of levocetirizine dihydrochloride) was drawn from the vial using a syringe (manufactured by Becton, Dickinson and Company). The 2.3 mL of the levocetirizine dihydrochloride solution was then injected into the open pump bottle containing the lotion base and three (3) 3.5 mm stainless steel balls. The levocetirizine dihydrochloride solution remaining in the vial was discarded.

0.6 mL of the cyanocobalamin solution (containing 0.035 g of cyanocobalamin) was drawn from the vial using a syringe (manufactured by Becton, Dickinson and Company). The 0.6 mL of cyanocobalamin solution was injected into the open pump bottle containing the lotion base and three (3) 3.5 mm stainless steel balls. The cyanocobalamin solution remaining in the vial was discarded.

3.0 mL of the mupirocin solution (containing 1 g of mupirocin) was drawn from the vial using a syringe (manufactured by Becton, Dickinson and Company). The 3.0 mL of mupirocin solution was injected into the open pump bottle containing the lotion base and three (3) 3.5 mm stainless steel balls. The mupirocin solution remaining in the vial was discarded.

An amount of lotion base from the syringe labelled “Replacement Lotion Base 12.07 g/12.07% for qs”, equivalent to the amount of drug and solvents not added to the bottle and discarded (8.1 mL, equivalent to 6.04 g), was added to the jar. The replacement lotion base remaining in the syringe was discarded.

A pump cap was installed onto the bottle containing the lotion base and solubilized active ingredients and tightened to seal the bottle. The bottle was shaken continuously for one (1) minute while tapping the bottom of the bottle firmly on the palm of the hand.

### Example 4

### Preparation of a Dermatological Lotion at Quarter Strength for all Active Ingredients Including Levocetirizine Dihydrochloride and Fluticasone Propionate

### Preparation of Primary Base Container

A lotion base was prepared by mixing 69.3 g Cetaphil® moisturizing cream (marketed by Galderma Laboratories), 0.70 g of xanthan gum (marketed by CP Kelco Co. as Xantural® 75), and 30 g of purified water with a Lightnin LabMaster G3U05R (SPX Corporation) overhead mixer. 88.9 g of the prepared lotion base was filled into a pump jar.

### Preparation of Active Agent Containers

A fixed amount of levocetirizine dihydrochloride (distributed by Professional Compounding Centers of America), equivalent to the maximum desired strength (2 g or 2% of the final product), was filled into a vial and a cap with a rubber stopper was applied thereto with a crimper. The vial was appropriately labelled as “Levocetirizine Dihydrochloride 2 g/2%.”

A fixed amount of fluticasone propionate (distributed by Glopec International Inc.), equivalent to the maximum desired strength (0.1 g or 0.1% of the final product), was filled into a vial and a cap with a rubber stopper was applied thereto with a crimper. The vial was appropriately labelled as “Fluticasone Propionate 0.1 g/0.1%”.

### Preparation of Non-Base Inactive Containers

A fixed amount of purified water, in an amount sufficient to solubilize the levocetirizine dihydrochloride (3 g or 3% of the final product), was filled into a syringe (manufactured by Becton, Dickinson and Company) with a Luer lock fitting and a cap was applied. The syringe was appropriately labelled as “Purified Water 3 g/3% for Levocetirizine Dihydrochloride”.

A fixed amount of benzyl alcohol, in an amount sufficient to solubilize the fluticasone propionate (6 g or 6% of the final product), was filled into a syringe (manufactured by Becton, Dickinson and Company) with a Luer lock fitting and a cap was applied. The syringe was appropriately labelled as “Benzyl Alcohol 6 g/6% for Fluticasone Propionate”.

A fixed amount of 18% sodium hydroxide solution, in an amount sufficient to neutralize final product of (0.675 g or 0.675% of the final product), was filled into a syringe (manufactured by Becton, Dickinson and Company). The syringe was appropriately labelled as “18% sodium hydroxide solution”.

### Preparation of Replacement Base Container

A fixed amount of the aforementioned lotion base, in an amount sufficient to replace all of the active ingredients and active solubilizing fluids (11.1 g or 11.1% of the final product), was filled into a syringe (manufactured by Becton, Dickinson and Company) with a Luer lock fitting. A cap was then applied to the syringe. The syringe was appropriately labelled as “Replacement Lotion Base 11.1 g/11.1% for qs”.

### Mixing of Active Agents, Non-Base Agents, and Base Composition

A needleless vial adapter (marketed by Yukon Medical as a 20 mm Vented ViaLok®) was installed onto the vial containing levocetirizine dihydrochloride. The syringe labelled “Purified Water 3 g/3% for Levocetirizine Dihydrochloride” was installed onto the needleless vial adapter. The purified water was injected into the vial and the contents of the vial were mixed by shaking the vial and by using the syringe to draw and dispense the contents until all of the levocetirizine dihydrochloride was solubilized. An amount of levocetirizine dihydrochloride solution containing 0.5 g of levocetirizine dihydrochloride (1.2 mL of the solution, equivalent to 1.25 g of the solution) was then drawn from the vial using the syringe. The 1.2 mL of levocetirizine dihydrochloride solution was injected into the open pump jar containing the lotion base. The levocetirizine dihydrochloride solution remaining in the vial was discarded.

A needleless vial adapter (marketed by Yukon Medical as a 20 mm Vented ViaLok®) was installed onto the vial containing fluticasone propionate 0.1 g/0.1% of the final product. The syringe labelled “Benzyl Alcohol 6 g/6% for Fluticasone Propionate” was installed onto the needleless vial adapter. The benzyl alcohol was injected into the vial and the contents of the vial were mixed by shaking the vial and by using the syringe to draw and dispense the contents until all of the fluticasone propionate is solubilized. An amount of fluticasone propionate solution containing 0.025 g of fluticasone propionate (1.5 mL of the solution, equivalent to 1.53 g of the solution) was then drawn from the vial using the syringe. The 1.5 mL of fluticasone propionate solution was injected into the open pump jar containing the cream base. The fluticasone propionate solution remaining in the vial was discarded.

The syringe containing the 18% sodium hydroxide solution was emptied into the pump jar containing the cream base.

An amount of replacement base from the syringe labelled “Replacement Lotion Base”, equivalent to the amount of drug and solvents not added to the jar and discarded (11.1 mL, equivalent to 7.645 g), was added to the open pump jar. The replacement base remaining in the syringe was discarded.

A mechanical stirrer is used to stir the cream base, levocetirizine dihydrochloride solution and fluticasone propionate solution mixture for a period of one (1) minute to form a cream product. A pump cap was installed onto the jar containing the compounded pharmaceutical product and tightened to seal the bottle. The bottle was shaken continuously for thirty (30) seconds while tapping the bottom of the bottle firmly on the palm of the hand.

### Example 5

### Preparation of a Gel Base

A gel base was prepared by weighing 66.25 g of purified water into a suitable stainless steel container. The contents of the container were mixed using a Lightnin LabMaster G3U05R (SPX Corporation) overhead mixer. 1 g of Carbomer Interpolymer Type A (marketed by Lubrizol as Carbopol® Ultrez 10 NF) was added slowly to the purified water and mixed therewith for 10 minutes. 0.5 g of triethanolamine (marketed by Sigma-Aldrich) was added to the mixture to adjust the pH and form a gel. 32.255 g of polyethylene glycol 400 (distributed by Sigma-Aldrich) and 0.095 g of sodium sulfite (distributed by Sigma-Aldrich) were added to the gel with mixing and mixed for 10 minutes.

### Example 6

### Preparation of a Pain Management Gel at Half Strength for all Active Ingredients Including Lidocaine and Prilocaine

### Preparation of Primary Base Container

A gel base was prepared using the formula and process described in Example 5. 76 g of the prepared gel base was filled into a pump jar.

### Filling of Prilocaine and Lidocaine Pouch

A three-chambered pouch was provided. The pouch contained a Luer lock fitting at one end. The Luer lock fitting is connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of prilocaine (distributed by Letco Medical), equivalent to the maximum desired strength (2.0 g or 2.0% of the final product), was filled into the chamber of the pouch which contained the Luer lock fitting. The chamber was appropriately labelled as “Prilocaine 2.0 g/2.0%”.

A fixed amount of lidocaine (distributed by Medisca Pharmaceutique Inc.), equivalent to the maximum desired strength (2.0 g or 2.0% of the final product), was filled into the middle chamber of the pouch. The chamber was appropriately labelled as “Lidocaine 2.0 g/2.0%”.

A 10:90 benzyl alcohol:propylene glycol solution was prepared by weighing 90 g of propylene glycol into a suitable stainless steel container. Mixing was then conducted using a Lightnin LabMaster G3U05R (SPX Corporation) overhead mixer to create a vortex. 10 g of benzyl alcohol (distributed by Sigma Aldrich Inc.) was added with continued mixing. The solution was then mixed for 15 minutes.

A fixed amount of the 10:90 benzyl alcohol:propylene glycol solution, in an amount sufficient to solubilize the lidocaine and prilocaine (20 g or 20% of the final product), was filled into the third chamber of the pouch. The chamber was appropriately labelled as “Benzyl Alcohol 10%/Propylene Glycol 90% Solution for Prilocaine and Lidocaine”.

### Preparation of Replacement Base Container

A fixed amount of propylene glycol (distributed by Sigma-Aldrich), in an amount sufficient to replace all of the active ingredients and active solubilizing fluids (24 g or 24% of the final product), was filled into a syringe (manufactured by Becton, Dickinson and Company) with a Luer lock fitting. A cap was then applied to the syringe. The syringe was appropriately labelled as “Replacement Propylene Glycol 24 g/24% for qs”.

### Mixing of Active Agents, Non-Base Agents, and Base Composition

Sufficient pressure was applied to the chamber containing the 10:90 benzyl alcohol:propylene glycol solution in the pouch to rupture the seals between the three chambers, allowing the solution to mix with the lidocaine and prilocaine. The pouch was kneaded between the fingers and thumbs for 1 minute until the lidocaine and prilocaine formed a miscible liquid with the benzyl alcohol:propylene glycol solution. A graduated syringe (manufactured by Becton, Dickinson and Company) was attached to the Luer lock fitting on the pouch. An amount of the liquid containing 1.0 g of prilocaine and 1.0 g of lidocaine (11.63 mL of the liquid, equivalent to 12.0 g of the liquid) was then drawn from the pouch using the syringe and injected into the pump jar containing the gel base.

An amount of propylene glycol contained in the syringe labelled “Replacement Propylene Glycol 24 g/24% for qs”, equivalent to the weight of drug and solvents not added to the jar and discarded (11.6 mL, equivalent to 12.0 g), was then introduced into the gel base in the pump jar. The propylene glycol remaining in the syringe was discarded.

A plastic spatula was used to stir the gel base, lidocaine and prilocaine solution and propylene glycol for a period of one (1) minute to form a gel product. A pump cap was installed onto the jar containing the cream product and tightened to seal the jar. The jar was then shaken continuously for thirty (30) seconds while tapping the bottom firmly on the palm of the hand.

### Example 7

### Preparation of a Dermatological Cream at Full Strength for all Active Ingredients Including Tacrolimus Monohydrate and Fluticasone Propionate

### Preparation of Primary Base Container

96.9 g of Vanicream™ moisturizing skin cream (marketed by Pharmaceutical Specialties, Inc.) was filled into a pump jar.

### Preparation of Active Agent Containers

A fixed amount of tacrolimus monohydrate (distributed by Teva Active Pharmaceutical Ingredients Division), equivalent to the maximum desired strength (0.05 g or 0.05% of the final product), was filled into a vial. A cap with a rubber stopper was applied onto the vial with a crimper. The vial was appropriately labelled as “Tacrolimus Monohydrate 0.05 g/0.05%”.

A fixed amount of fluticasone propionate (distributed by Teva Active Pharmaceutical Ingredients Division), equivalent to the maximum desired strength (0.05 g or 0.05% of the final product), was filled into a vial. A cap with a rubber stopper was applied to the vial with a crimper. The vial was appropriately labelled as “Fluticasone Propionate 0.05 g/0.05%”.

### Preparation of Non-Base Inactive Containers

A fixed amount of benzyl alcohol (distributed by Sigma-Aldrich), in an amount sufficient to solubilize the tacrolimus monohydrate (1 g or 1% of the final product), was filled into a syringe (manufactured by Becton, Dickinson and Company) with a Luer lock fitting. A cap was applied. The syringe was appropriately labelled as “Benzyl Alcohol 1 g/1% for Tacrolimus Monohydrate”.

A fixed amount of benzyl alcohol (distributed by Sigma-Aldrich), in an amount sufficient to solubilize the fluticasone propionate (2 g or 2% of the final product), was filled into a syringe (manufactured by Becton, Dickinson and Company) with a Luer lock fitting. A cap was applied. The syringe was appropriately labelled as “Benzyl Alcohol 2 g/2% for Fluticasone Propionate”.

### Preparation of Replacement Base Container

A fixed amount of the aforementioned cream, in an amount sufficient to replace all of the active ingredients and active solubilizing fluids (6.1 g or 6.1% of the final product), was filled into a syringe (manufactured by Becton, Dickinson and Company) with a Luer lock fitting. A cap was then applied to the syringe. The syringe was appropriately labelled as “Replacement Cream Base 6.1 g/6.1% for qs”.

### Mixing of Active Agents, Non-Base Agents, and Base Composition

A needleless vial adapter (marketed by Yukon Medical as a 20 mm Vented ViaLok®), was installed onto the vial containing tacrolimus monohydrate. The syringe labelled “Benzyl Alcohol 1 g/1% for Tacrolimus Monohydrate” was installed onto the needleless vial adapter. The benzyl alcohol was injected into the vial and the contents of the vial mixed by shaking the vial and by using the syringe to draw and dispense the contents until all of the tacrolimus monohydrate was solubilized. An amount of tacrolimus monohydrate solution, containing 0.05 g of tacrolimus monohydrate (0.95 mL of the solution, equivalent to 1.05 g of the solution), was then drawn from the vial using the syringe. The 0.95 mL of tacrolimus monohydrate solution was injected into the open pump jar containing the cream base. The tacrolimus monohydrate solution remaining in the vial was discarded.

A needleless vial adapter (marketed by Yukon Medical as a 20 mm Vented ViaLok®) was installed onto the vial containing fluticasone propionate 0.05 g/0.05% of the final product. The syringe labelled “Benzyl Alcohol 2 g/2% for Fluticasone Propionate” was installed onto the needleless vial adapter. The benzyl alcohol was injected into the vial and the contents of the vial mixed by shaking the vial and by using the syringe to draw and dispense the contents until all of the fluticasone propionate was solubilized. An amount of fluticasone propionate solution, containing 0.05 g of fluticasone propionate (2.0 mL of the solution, equivalent to 2.05 g of the solution) was then drawn from the vial using the syringe. The 3.35 mL of fluticasone propionate solution was injected into the open pump jar containing the cream base. The fluticasone propionate solution remaining in the vial was discarded.

The replacement cream base labelled “Replacement Cream Base” in the syringe was discarded.

Three (3) 3.5 mm stainless steel balls were inserted into the jar and a pump cap was installed onto the jar containing the compounded pharmaceutical product and tightened to seal the jar. The jar was shaken continuously for thirty (30) seconds while tapping the bottom of the jar firmly on the palm of the hand.

### Example 8

### Preparation of a Wound Care Ointment at Full Strength for Collagenase and Half Strength for Mupirocin

### Preparation of Primary Base Container

An ointment base was prepared by heating 20 g of polyethylene glycol 1450 (marketed by Dow Chemical Co. as CARBOWAX™ Polyethylene Glycol (PEG) 1450) with 80 g polyethylene glycol 400 (marketed by The Dow Chemical Co. as CARBOWAX™ Polyethylene Glycol (PEG) 400) along with mixing using a Lightnin LabMaster G3U05R (SPX Corporation) overhead mixer. 74.86 g of the prepared ointment base was filled into one chamber of a pump container containing two chambers.

### Filling of Collagenase Pouch

A two-chambered pouch was provided. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of collagenase (distributed by Worthington Biochemical Corp.), equivalent to the maximum desired strength (350 U/g) (0.14 g or 0.14% of the final product), was filled into the a chamber of the pouch which contained the Luer lock fitting. The chamber of the pouch was appropriately labelled as “Collagenase 0.14 g/0.14%”.

A fixed amount of purified water, in an amount sufficient to solubilize the collagenase (5 g or 5% of the final product), was filled into the second chamber of the two-chambered pouch. The pouch chamber was appropriately labelled as “Purified water 5 g/5% for Collagenase”.

### Filling of Mupirocin Pouch

A two-chambered pouch was provided. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of mupirocin (distributed by Teva Active Pharmaceutical Ingredients Division), equivalent to the maximum desired strength (2 g or 2% of the final product), was filled into the a chamber of the pouch which contained the Luer lock fitting. The chamber of the pouch was appropriately labelled as “Mupirocin 2 g/2%”.

A fixed amount of propylene glycol, in an amount sufficient to solubilize the mupirocin, (18 g or 18% of the final product) was filled into the second chamber of the pouch. The pouch chamber was appropriately labelled as “Propylene Glycol 18 g/18% for Mupirocin”.

### Preparation of Replacement Base Container

A fixed amount of propylene glycol, in an amount sufficient to replace all of the active ingredients and active solubilizing fluids (25.14 g or 25.14% of the final product), was filled into a syringe (manufactured by Becton, Dickinson and Company) with a Luer lock fitting. A cap was then applied to the syringe. The syringe was appropriately labelled as “Replacement Propylene Glycol 25.14 g/25.14% for qs”.

### Mixing of Active Agents, Non-Base Agents, and Base Composition

Sufficient pressure was applied to the pouch chamber containing purified water in the pouch containing collagenase to rupture the seal between the two chambers therein, allowing the purified water to mix with the collagenase. The pouch was kneaded between the fingers and thumbs for 1 minute until the collagenase was dissolved resulting in a clear solution. A syringe was attached to the Luer lock fitting on the pouch and used to withdraw all of the solution (5.14 g) therefrom. The solution was then introduced into the remaining empty chamber of the two chamber pump container.

Sufficient pressure was applied to the pouch chamber containing propylene glycol in the pouch containing mupirocin to rupture the seal between the two chambers therein, allowing the solution to mix with the mupirocin. The pouch was kneaded between the fingers and thumbs for 1 minute until the mupirocin formed a clear solution with the propylene glycol. A graduated syringe was attached to the Luer lock fitting on the pouch and used to withdraw all of the solution therein. Half of the solution (9.6 mL of the solution, equivalent to 10 g of the solution) was then also introduced into the chamber of the two chamber pump container which contained collagenase. The mupirocin solution remaining in the syringe was discarded.

An amount of propylene glycol equivalent to the discarded mupirocin solution (9.6 mL, equivalent to 10 g) from the syringe labelled as “Replacement Propylene Glycol 25.14 g/25.14% for qs” was added to the chamber of the two chamber pump container containing collagenase and mupirocin. The propylene glycol remaining was discarded.

A pump cap containing a static mixer was installed onto the two chamber pump container containing the ointment product and tightened to seal the bottle. The container was shaken continuously for thirty (30) seconds while tapping the bottom thereof firmly on the palm of the hand. The resulting collagenase and mupirocin solution in the first chamber of the two chamber pump container mixed at the proper ratio (25.14:74.86) with the ointment base in the second chamber of the pump container when the pump mechanism was depressed. As a result, a collagenase and mupirocin ointment having uniformity is dispensed.

### Example 9

### Preparation of a Wound Care Gel at Full Strength for Tobramycin and Lidocaine and Half Strength for Phenytoin Sodium

### Gel Base

A gel base was prepared by first weighing 15.7 g of purified water into a suitable stainless steel container. Mixing was initiated using a Lightnin LabMaster G3U05R (SPX Corporation) overhead mixer. 0.4 g of sodium sulfite (distributed by Sigma-Aldrich) was added to the purified water and mixed until dissolved. 1.8 g of polyvinylpyrrolidone (marketed by BASF as Kollidon® 90F) was then added slowly to the solution with mixing for 5 minutes. 1.8 g of polyoxyl 40 hydrogenated castor oil (marketed by BASF as Kolliphor™ RH 40) was then added to the solution and mixed for 2 minutes. A premixed slurry was made by dispersing 1.8 g of hydroxyethyl cellulose (marketed by Ashland as Natrosol™ 250 HHX Pharm) in 9.2 g of propylene glycol (marketed by Sigma-Aldrich). This premixed slurry was added slowly to the dispersion and mixed for 2 minutes. 69.3 g of propylene glycol was added to the dispersion and mixed for an additional 10 minutes. The dispersion was then homogenized for 10 minutes, using a labscale Silverson Overhead Homogenizer L-5M-A with a one inch tubular head, to form a gel.

### Preparation of Primary Base Container

The gel base prepared was used to prepare the final gel product. 54.5 g of the prepared gel base was filled into a pump jar.

### Filling of Tobramycin Pouch

A two-chambered pouch was used. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of tobramycin (distributed by Professional Compounding Centers of America), equivalent to the maximum desired strength (5 g or 5% of the final product), was filled into the chamber of the pouch containing the Luer lock fitting and the chamber was sealed using a heat sealer. The chamber of the pouch was appropriately labelled as “Tobramycin 5 g/5%”.

A fixed amount of purified water, in an amount sufficient to solubilize the tobramycin (7.5 g or 7.5% of the final product) was filled into the second chamber of the pouch and the chamber was sealed using a heat sealer. The chamber was appropriately labelled as “Purified water 7.5 g/7.5% for Tobramycin”.

### Filling of Phenytoin Pouch

A two-chambered pouch was provided. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of phenytoin sodium (distributed by Professional Compounding Centers of America), equivalent to the maximum desired strength (5 g or 5% of the final product), was filled into the chamber of the pouch containing the Luer lock fitting and the chamber was sealed using a heat sealer. The chamber of the pouch was appropriately labelled as “Phenytoin sodium 5 g/5%”.

A fixed amount of propylene glycol, in an amount sufficient to suspend the phenytoin sodium (16 g or 16% of the final product), was filled into the second chamber of the pouch. The chamber was appropriately labelled as “Propylene Glycol 16 g/16% for Phenytoin”.

### Filling of Lidocaine Pouch

A two-chambered pouch was provided. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of lidocaine (distributed by Medisca Inc.), equivalent to the maximum desired strength (2 g or 2% of the final product) was filled into the first chamber of the pouch containing the Luer lock fitting and the chamber was sealed using a heat sealer. The chamber of the pouch was appropriately labelled as “Lidocaine 2 g/2%”.

A fixed amount of propylene glycol, in an amount sufficient to solubilize the lidocaine (10 g or 10% of the final product), was filled into the second chamber of the pouch and the chamber was sealed using a heat sealer. The chamber was appropriately labelled as “Propylene glycol 10 g/10% for Lidocaine”.

### Preparation of Replacement Base Container

A fixed amount of propylene glycol, in an amount sufficient to replace all of the above active and non-base inactive ingredients (45.5 g or 45.5% of the final product), was filled into a syringe (manufactured by Becton, Dickinson and Company) with a Luer lock fitting. A cap was then applied to the syringe. The syringe was appropriately labelled as “Replacement Propylene Glycol 45.5 g/45.5% for qs”.

### Mixing of Active Agents, Non-Base Agents, and Base Composition

Sufficient pressure was applied to the pouch chamber containing the purified water in the pouch containing tobramycin to rupture the seal between the two chambers, allowing the purified water to mix with the tobramycin. The pouch was kneaded between the fingers and thumbs for 1 minute until the tobramycin was dissolved resulting in a clear solution. A graduated syringe (manufactured by Becton, Dickinson and Company) was attached to the Luer lock fitting and used to withdraw the solution. All of the solution was then drawn from the pouch using the syringe and injected into the pump jar containing the gel base.

Three (3) 3.2 mm stainless steel balls were inserted into the jar containing the gel product and a pump cap was installed and tightened to seal the jar. The jar was then shaken continuously for thirty (30) seconds while tapping the bottom firmly on the palm of the hand.

Sufficient pressure was applied to the pouch chamber containing the propylene glycol in the pouch containing phenytoin sodium to rupture the seals between the two chambers, allowing the propylene glycol to mix with the phenytoin sodium. The pouch was kneaded between the fingers and thumbs for 1 minute until the phenytoin sodium formed a clear solution with the propylene glycol. All of the solution was withdrawn from the pouch by attaching a graduated syringe (manufactured by Becton, Dickinson and Company) attached to the Luer lock fitting. The pump cap of the pump jar containing the tobramycin gel was uninstalled. Half of the solution (9.5 mL of the solution, equivalent to 10.5 g of the solution) was then introduced into the pump jar. The remaining phenytoin solution in the syringe was discarded. The cap was reinstalled and tightened to seal the jar. The jar was then shaken continuously for thirty (30) seconds while tapping the bottom firmly on the palm of the hand.

Sufficient pressure was applied to the pouch chamber containing propylene glycol in the pouch containing lidocaine to rupture the seals between the two chambers, allowing the propylene glycol to mix with the lidocaine. The pouch was kneaded between the fingers and thumbs for 1 minute until the lidocaine formed a solution with the propylene glycol. A graduated syringe (manufactured by Becton, Dickinson and Company) was attached to the Luer lock fitting and used to withdraw all of the solution from the pouch. The pump cap of the jar containing the tobramycin and phenytoin gel was uninstalled. All of the lidocaine solution was then introduced into the pump jar.

An amount of propylene glycol equivalent to the discarded phenytoin solution (10.1 mL, equivalent to 10.5 g) was added to the pump jar containing the gel product. The remaining contents of the syringe labelled as “Replacement Propylene Glycol 45.5 g/45.5%” were discarded. The pump was reinstalled and tightened to seal the jar. The jar was then shaken continuously for thirty (30) seconds while tapping the bottom firmly on the palm of the hand.

### Example 10

### Preparation of a Suppository at Full Strength for Nifedipine and Half Strength Lidocaine for Use in Controlling Anal Fissures

### Preparation of Primary Base

72.8 g of suppository base was prepared according to the formula and process outlined below.

400 g of water was weighed into an appropriately-sized stainless steel beaker. The beaker was set on hot/stir plate with heat on to form a water bath. Heating was initiated.

69.16 g of polyethylene glycol (PEG) 1000 (marketed by DOW Chemical Co. as CARBOWAX™ polyethylene glycol 1000) was weighed into an appropriately sized stainless steel beaker. The beaker was placed in the water bath. Mixing was initiated using a Lightnin LabMaster G3U05R (SPX Corporation) overhead mixer. 3.64 g of polyethylene glycol (PEG) 3350 (marketed by DOW Chemical Co. as CARBOWAX™ polyethylene glycol 3350) was added to the polyethylene glycol 1000 with continued mixing. The mixture was mixed with the Lightnin LabMaster G3U05R (SPX Corporation) overhead mixer with continued heating of water bath. The mixture was mixed until the temperature reached 55-62° C. The mixture, which formed a suppository base, was removed from water bath and allowed to cool to room temperature. The water from the water bath was discarded.

### Preparation of Heat Resistant Glass Jar Container

72.8 g of the prepared suppository base was transferred into a heat resistant jar. The jar was labelled “Polyethylene Glycol Base for Suppositories”.

### Filling of Nifedipine Pouch

A two-chambered pouch was provided. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of nifedipine (distributed Teva Active Pharmaceutical Ingredients Division), equivalent to the maximum desired strength (0.2 g or 0.2% of the final product), was filled into the chamber of the pouch that contained the Luer lock fitting. The chamber of the pouch was appropriately labelled as “Nifedipine 0.2 g/0.2%”.

A fixed amount of polyethylene glycol 300 (marketed by BASF), in an amount sufficient to solubilize the nifedipine (5 g or 5% of the final product), was filled into the second chamber of the pouch. The chamber was appropriately labelled as “Polyethylene glycol 300 5 g/5% for Nifedipine”.

### Filling of Lidocaine Pouch

A two-chambered pouch was provided. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of lidocaine (distributed by Medisca Pharmaceutique Inc., equivalent to the maximum desired strength (2 g or 2% of the final product), was filled into the chamber of the pouch that contained the Luer lock fitting. The chamber of the pouch was appropriately labelled as “Lidocaine 2 g/2%”.

A fixed amount of polyethylene glycol 300, in an amount sufficient to solubilize the lidocaine (20 g or 20% of the final product), was filled into the second chamber of the pouch. The chamber was appropriately labelled as “Polyethylene glycol 300 20 g/20% for Lidocaine”.

### Preparation of Replacement Base Container

A fixed amount of polyethylene glycol 300, in an amount sufficient to replace all of the above active ingredients and active solubilizing fluids (27.2 g or 27.2% of the final product), was filled into a syringe (manufactured by Becton, Dickinson and Company) with a Luer lock fitting. A cap was then applied to the syringe. The syringe was appropriately labelled as “Replacement Polyethylene Glycol 300 27.2 g/27.2% for qs”.

### Mixing of Active and Inactive Agents

Sufficient pressure was applied to the pouch chamber containing the polyethylene glycol 300 in the pouch containing nifedipine to rupture the seal between the two chambers, allowing the polyethylene glycol 300 to mix with the nifedipine. The pouch was kneaded between the fingers and thumbs for 1 minute until the nifedipine was solubilized and the solution was uniform. A graduated syringe (manufactured by Becton, Dickinson and Company) was attached to the Luer lock fitting on the pouch and used to withdraw all of the solution therein. The solution was held until ready for further processing.

Sufficient pressure was applied to the pouch chamber containing the polyethylene glycol 300 in the pouch containing lidocaine to rupture the seal between the two chambers, allowing the polyethylene glycol 300 to mix with the lidocaine. The pouch was kneaded between the fingers and thumbs for 1 minute until the lidocaine was solubilized and the solution was uniform. A graduated syringe (manufactured by Becton, Dickinson and Company) was attached to the Luer lock fitting on the pouch and used to withdraw all of the solution therein. The solution was held until ready for further processing.

The heat resistant glass jar containing 72.8 g of polyethylene glycol base labelled “Polyethylene Glycol Base for Suppositories” was placed on a hot plate and heated mildly until the polyethylene glycol bas was melted. The nifedipine solution prepared earlier was transferred from the syringe into the heated jar while continuing to heat mildly. Half of the lidocaine solution, containing 1.0 g of lidocaine (9.5 mL of the solution, equivalent to 11.0 g of the solution), was then transferred from the syringe into the heated jar while continuing to heat mildly. The remaining lidocaine solution was discarded. An amount of polyethylene glycol 300, from the syringe, labelled as “Replacement Polyethylene Glycol 300 27.2 g/27.2% for qs”, equivalent to the discarded lidocaine suspension (9.5 mL or 11.0 g), was then added to the heated jar while continuing to heat mildly. The remaining contents of the syringe labelled as “Replacement Polyethylene Glycol 300 27.2 g/27.2%” were discarded.

The molten mixture was then stirred with a plastic stirrer for 1 minute until uniform while continuing to heat mildly. The molten mixture was then poured into 36 suppository molds (12 molds per strip supplied by Health Care Logistics Inc.). Excess material at the top of the molds was scraped away with a plastic spatula. The filled molds were then cooled at room temperature and filled into a dispensing package.

### Example 11

### Preparation of Pain Management Capsules at Half Strength for Naltrexone Hydrochloride

### Preparation of the Primary Base and Replacement Base Containers

A predetermined amount (12.69 g) of lactose monohydrate (distributed by the Professional Compounding Centers of America) was filled into a sachet containing a tear off opening. The first three sides of the sachet were sealed prior to filling and the fourth side was sealed after filling. The sachet was labelled “Lactose Monohydrate for Naltrexone Hydrochloride”.

1.25 g of lactose monohydrate was filled into each of four smaller similar sachets. The first three sides of the sachets were sealed prior to filling and the fourth side was sealed after filling. The sachets were each labeled as “Replacement Lactose Monohydrate 1.25 g for qs”.

### Filling of Naltrexone Hydrochloride Sachets

1.25 g of naltrexone hydrochloride (supplied by Mallinckrodt) was filled into each of four individual sachets. Each sachet was labelled “Naltrexone Hydrochloride 1.25 g.”

### Preparation of Capsule Powder for Half Strength Naltrexone Hydrochloride

Two of the sachets (for a half dose of 25 mg per capsule) labelled “Naltrexone Hydrochloride 1.25 g” were emptied into a mortar. An amount of lactose monohydrate approximately equal to the 1.25 g of naltrexone hydrochloride was added to the mortar from the sachet labelled “Lactose Monohydrate for Naltrexone Hydrochloride”. The combination was triturated using a pestle for 30 seconds. An additional amount of lactose monohydrate, approximately equal to the mixture in the mortar (2.5 g) was added to the mortar. The combination was triturated using a pestle for an additional 30 seconds. An additional amount of lactose monohydrate, approximately equal to the mixture in the mortar (5 g) was added to the mortar. The combination was triturated using a pestle for an additional 30 seconds. The remainder of the lactose monohydrate from the sachet labeled “Lactose Monohydrate for Naltrexone Hydrochloride” was added to the mortar and triturated for an additional 30 seconds. Since only two of the four sachets of naltrexone hydrochloride were added, two of the “Replacement Lactose Monohydrate 1.25 g” were added and a final trituration was done.

### Punching of Capsules

The triturated powder was placed on one side of a sheet of paraffin wax paper in an even layer approximately the depth of a 3 capsule body. The second half of the paraffin wax paper was folded over onto the powder and pressure was applied by hand to slightly compact the powder. A capsule body was pressed into the compressed powder bed repeatedly until the capsule body was full. Capsule lids were placed onto the capsule bodies. This process was repeated to form 100 capsules. Each capsule was weighed to confirm the weight. The capsules were placed into a vial and appropriately labeled.

### Example 12

### Preparation of a Gel at Full Strength for all Active Ingredients Including Benzocaine and Lidocaine for Use as a Topical Anesthetic

### Preparation of a Gel Base

A gel base was prepared by weighing 39.6 g of propylene glycol (distributed by BASF as Kollisolv® PG) into a suitable stainless steel container. Mixing was initiated using a Lightnin LabMaster G3U05R (SPX Corporation) overhead mixer. 2.0 g of hydroxyethyl cellulose (marketed by Ashland as Natrosol 250 HHX) was added to the propylene glycol, and mixed for 5 minutes. 10.0 g purified water was weighed into a separate suitable stainless steel container. A stir bar was placed in the container and mixing was initiated on an unheated stir plate. 0.30 g of sodium sulfite (distributed by Sigma-Aldrich) was added to the mixing water. The stir bar was removed from the water mixture and the solution was added to the propylene glycol solution with mixing to form a gel and was mixed for 10 minutes. 1.1 g of 88% lactic acid (distributed by Archer Daniels Midland Company) was then added to the gel and mixed for 10 minutes with the Lightnin LabMaster G3U05R (SPX Corporation) overhead mixer.

### Preparation of Primary Base Container

A gel base was prepared using the formula and process described above and used to formulate this gel product. 53 g of the prepared gel base is filled into a pump jar.

### Filling of Benzocaine Pouch

A two-chambered pouch was provided. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of benzocaine (distributed by Sigma-Aldrich), equivalent to the maximum desired strength (10 g or 10% of the final product), was filled into the chamber of the pouch containing the Luer lock fitting. The chamber of the pouch was appropriately labelled as “Benzocaine 10 g/10%”.

A fixed amount of polyethylene glycol 300 (distributed by BASF as Pluriol E 300 NF), in an amount sufficient to solubilize the benzocaine, (22 g or 22% of the final product) was filled into the second chamber of the pouch. The chamber was appropriately labelled as “Polyethylene Glycol 300 22 g/22% for Benzocaine”.

### Filling of Lidocaine Pouch

A two-chambered pouch was provided. The pouch contained a Luer lock fitting which also connected to the interior of one of the internal chambers. Each chamber had three sides. The first three sides of each chamber in the pouch were sealed prior to filling with the fourth side being sealed after filling. The sides of the chambers internal within the pouch and adjoining another internal chamber were designed to rupture under pressure.

A fixed amount of lidocaine (distributed by Medisca Pharmaceutique Inc.), equivalent to the maximum desired strength (5 g or 5% of the final product), was filled into the chamber of the pouch that contained the Luer lock fitting. The pouch chamber was appropriately labelled as “Lidocaine 5 g/5%”.

A fixed amount of propylene glycol (distributed by BASF as Kollisolv® PG), in an amount sufficient to solubilize lidocaine (10 g or 10% of the final product), was filled into the second chamber of the pouch. The chamber was appropriately labelled as “Propylene glycol 10 g/10% for Lidocaine”.

Preparation of Replacement Base Container

A fixed amount of propylene glycol (distributed by BASF as Kollisolv® PG, in an amount sufficient to replace all of the active ingredients and active solubilizing fluids, (47.0 g or 47.0% of the final product) was filled into a syringe with a Luer lock fitting. A cap was then applied to the syringe. The syringe was appropriately labelled as “Replacement propylene glycol 47.0 g/47.0% for qs”.

### Mixing of Active Agents, Non-Base Agents, and Base Composition

Sufficient pressure was applied to the pouch chamber containing the polyethylene glycol 300 in the pouch containing benzocaine to rupture the seal between the two chambers, allowing the solution to mix with benzocaine. The pouch was kneaded between the fingers and thumbs for 1 minute until the benzocaine was dissolved resulting in a clear solution. A graduated syringe (manufactured by Becton, Dickinson and Company) was attached to the Luer lock fitting on the pouch and used to withdraw the entire solution. An amount of the benzocaine solution containing 10 g of benzocaine (28 mL of the solution, equivalent to 32 g of the solution) was then injected into the pump jar containing the gel base.

Sufficient pressure was applied to the pouch chamber containing the propylene glycol in the pouch containing lidocaine to rupture the seals between the two chambers, allowing the solution to mix with the lidocaine. The pouch was kneaded between the fingers and thumbs for 1 minute until the lidocaine was dissolved resulting in a clear solution. A graduated syringe (manufactured by Becton, Dickinson and Company) was attached to the Luer lock fitting on the pouch and used to withdraw the entire solution. An amount of the lidocaine solution containing 5 g of Lidocaine (14.7 ml of the solution, equivalent to 15 g of the solution) was then injected into the pump jar.

Three (3) 3.2 mm stainless steel balls were inserted into the jar containing the gel product and a pump cap was installed and tightened to seal the jar. The jar was shaken continuously for sixty (60) seconds while tapping the bottom of the bottle firmly on the palm of the hand.

1. A kit for use in the preparation of a compounded pharmaceutical product, said kit comprising an active agent container, wherein said kit is capable of producing compounded pharmaceutical products wherein the pharmaceutically active agent can be present in said product at various dosage strengths.

2. The kit according to claim 1, wherein said kit further comprises an inactive agent container associated with said active agent container.

3. The kit according to claim 2, wherein said kit further comprises a connector means.

4. The kit according to claim 2, wherein said active agent container or said inactive agent container is in the form of a media dispenser which is capable of drawing and dispensing a metered amount of the contents therein.

5. The kit according to claim 2, wherein said kit comprises at least two inactive agent containers with one being a non-base inactive container and another being a base container, said non-base inactive container being in the form of a media dispenser.

6. The kit according to claim 2 wherein said inactive agent container and said active container are chambers within a multi-chambered pouch and wherein the barrier between said containers is frangible or contains a frangible element and said kit further comprises a recipient container which is capable of drawing and dispensing metered amounts of media.

7. The kit according to claim 6, wherein said kit comprises at least two inactive agent containers with one being a non-base inactive container contained in said pouch with said active container and another being a base container which is separate from said pouch.

8. The kit according to claim 6, further comprising a mixing means, a dispensing means, and/or a replacement base container.

9. The kit according to claim 1, wherein said kit further comprises an apparatus which comprises a housing which contains a chamber which serves as a base container and a chamber which serves as a recipient container, a mixing means, and a dispensing means.

10. The kit according to claim 1, wherein said kit further comprises a suppository mold.

11. The kit according to claim 1, comprising an active agent selected from the group consisting of: tobramycin, phenytoin, lidocaine, diclofenac, prilocaine, levofloxicin, nystatin, collagenase, hyaluronidase, mupirocin, levocetirizine, cyanocobalamin, calcipotriene, fluticasone, tacrolimus, sirolimus, ketamine, gabapentin, bupivacaine, cyclobenzaprine, fluorouracil, salicylic acid, nifedipine, itraconazole, undecylenic acid, naltrexone, benzocaine, and combinations thereof.

12. The kit according to claim 11, wherein said mixing means comprises solid particles that do not interact with the drug product and help to mix the product when shaken.

13. The kit according to claim 1 comprising two or more active agents.

14. The kit according to claim 1, wherein said kit allows for the production of multiple unit of use amounts of a compounded pharmaceutical product, with each unit of use amount having the same or different dosage strength.

15. The kit according to claim 1, wherein said kit is capable of producing a product having an Acceptance Value of less than 15 when 10 units thereof are tested.

16. A method for making a compounded pharmaceutical product using a kit according to claim 2, wherein said method comprises the step of mixing an active agent from said active agent container with an inactive agent contained in said inactive agent container.

17. A method for making a compounded pharmaceutical product using a kit according to claim 3, wherein said inactive agent container is connected to said active agent container by said connector means and the contents of said containers are mixed, then a recipient container in the form of a media dispenser is attached to said containers by way of said connector means and said media dispenser is used to draw and dispense said mixture, wherein the drawing of the mixture and/or the dispensing of the mixture is of the full amount or a smaller metered amount of the mixture.

18. A method for making a compounded pharmaceutical product using a kit according to claim 4, wherein said inactive agent container is in the form of a media dispenser and is connected to said active agent container and used to dispense the contents therein into the active agent container, forming a mixture of the active agent and the inactive agent or agents contained in the inactive agent container, and said media dispenser is then used to draw and dispense said mixture, wherein the drawing of the mixture and/or the dispensing of the mixture is of the full amount or a smaller metered amount of the mixture.

19. A method for making a compounded pharmaceutical product using a kit according to claim 6, wherein pressure is applied to said pouch to fragment the barrier or barriers between the chambers therein or the frangible element contained in said barrier or barriers, a recipient container is then attached to said pouch and used to draw and then dispense the contents of said pouch, wherein the drawing of the contents and/or the dispensing of the contents is of the full amount or a smaller metered amount of the contents.

20. A compounded pharmaceutical product made using the method according to claim 16.
